(19) United States
(12) Patent Application Publication
(10) Pub. No.: US 2016/0033504 A1
(43) Pub. Date: Feb. 4, 2016

(54) PROTOCOL FOR IDENTIFYING AND ISOLATING ANTIGEN-SPECIFIC B CELLS AND PRODUCING ANTIBODIES TO DESIRED ANTIGENS

(71) Applicant: ALDER BIOPHARMACEUTICALS, INC., Bothell, WA (US)

(72) Inventors: Daniel S. Allison, Lake Forest Park, WA (US); Benjamin H. Dutzar, Seattle, WA (US); Leon F. Garcia-Martinez, Woodinville, WA (US); Katie Anderson, Kirkland, WA (US); Ethan W. Ojala, Snohomish, WA (US); John A. Latham, Seattle, WA (US); Jens Billgren, Seattle, WA (US); Anne Elisabeth Carvalho Jensen, Snohomish, WA (US)

(21) Appl. No.: 14/776,945

(22) PCT Filed: Mar. 18, 2014
(86) PCT No.: PCT/US14/30994

§ 371 Date: Sep. 15, 2015

Related U.S. Application Data
(60) Provisional application No. 61/791,755, filed on Mar. 15, 2013.

(51) Int. Cl. G01N 33/569

(52) U.S. Cl. CPC G01N 33/56972

(57) ABSTRACT

Methods of identifying antigen-specific antibody-secreting and antibody-forming cells, such as antigen-specific B cells, and methods for cloning the antigen-specific antibody sequences of the antibody produced by these cells are provided. In particular, the methods include enriching B cells for antigen-specific B cells, culturing the antigen-specific B cells to generate clonal B cell populations, detecting clonal B cells that produce a single antigen-specific antibody, optionally screening the clonal B cell populations for functional activity, staining and sorting the cells to isolate the antigen-specific B cells, sequencing the nucleic acids encoding the antigen-specific antibody sequences, expressing the sequences to produce an antibody, isolating the antibody and screening the antibody for antigen recognition. The methods provide improved enrichment and selection of antigen-specific antibody-secreting and antibody-forming cells, which enhances recovery of antigen-specific antibodies.

### RELATED APPLICATION DISCLOSURE

This application is a U.S. National Phase Application submitted under 35 U.S.C. 371 based on International Application No. PCT/US14/30994 filed Mar. 18, 2014 (published as WO 2014-146074 on Sep. 18, 2014), which claims the benefit of U.S. Provisional Application Ser. No. 61/791,755, filed Mar. 15, 2013, each of which is hereby incorporated by reference in its entirety.

This application includes as part of its disclosure a biological sequence listing, contained in a file named “43257o3803.txt” created on Sep. 15, 2015 and having the size 3,751 bytes, which is hereby incorporated by reference in its entirety.

### FIELD OF THE INVENTION

The invention relates to methods of identifying antibody-secreting and antibody-forming cells, particularly rabbit antigen-specific B cells, and methods for cloning the antigen-specific sequences of the antibodies produced by these cells and methods for expressing variants of these antibody sequences, especially humanized and chimeric versions of these antibody sequences. The subject methods may be used to derive high quality antibodies to different antigens, e.g., human and viral polypeptides, as well as small peptides and other antigens that are relatively non-immunogenic and/or difficult to generate high quality antibodies using some other B cell selection methods.

### BACKGROUND OF THE INVENTION

There are known methods for generating monoclonal antibodies that are based on the isolation of B lymphocytes that produce antibodies targeting a particular antigen. These methods generally depend on the use of purified antigen or a mixture of antigens to identify and isolate B lymphocytes that bind that antigen (or antigens). Methods that depend on the use of antigen or mixtures of antigens to select antibody-forming cells (AFC) or B lymphocytes that express surface-receptors specific for an antigen, include using antigen-coated magnetic beads (Lagerkvist et al., 1995) or fluorochrome-labelled antigens and fluorescence activated cell-sorting (FACS) (Weitkamp et al., 2003) to isolate cells which have then been commonly expanded into clones. Monoclonal antibodies are then generated from these clones, for example by fusion to generate hybridomas (Steenbakkers et al., 1993) or by cloning of the genes encoding the antibody variable regions (e.g. using RT-PCR) (Lagerkvist et al., 1995; Wang & Stollar, 2000; Weitkamp et al., 2003).

Alternatively, methods have been described to identify individual cells that are secreting antibody specific for a particular antigen, including using a hemolytic plaque assay with antigen-coupled erythrocytes, after which techniques such as RT-PCR can be used to clone the genes encoding the antibody variable regions (Babcook et al., 1996; U.S. Pat. No. 5,627,052 (1997) Schrader, J. W.).

The present invention provides methods for identifying antibody-secreting cells (ASC) that have a high likelihood of secreting antibodies specific for a desired antigen (e.g., antigen-specific B cells) and generating ASC or clones of ASC from antibody-forming cells, and cloning the antigen-specific antibody variable sequences encoding the variable light chain region and/or the variable heavy chain region of the antibodies specific for a desired antigen that are secreted by these ASC. In particular, the methods include enrichment of antigen-specific ASC; a primary screening step for antigen-recognition; and optional screening for functional properties in combination with ASC staining and sorting to improve the yield of ASC, preferably antigen-specific B cells. The methods can be applied to the generation of monoclonal antibodies from any species that makes antibodies. In preferred embodiments the methods are effected using rabbit or human B cells.

### SUMMARY OF THE INVENTION

The present invention provides methods for identifying B cells that expresses an antigen-specific antibody (i.e., antigen-specific B cells), comprising: (i) obtaining B cells from a host that has been immunized or exposed naturally to an antigen of interest; (ii) enriching a fraction of said B cells to obtain an enriched population of antigen-specific B cells, i.e., which contain a greater percentage of B cells that produce an antibody that binds to the antigen of interest relative to the B cell fraction prior to enrichment; (iii) separately culturing one or more fractions from said enriched antigen-specific B cell population under culture conditions that favor the formation of a clonal B cell population that produces a single antibody that binds to the antigen of interest; (iv) detecting the clonal B cell population that produces a single antibody that binds to the antigen of interest, thereby identifying one or more antigen-specific B cells; (v) optionally screening the clonal antigen-specific B cell population identified in step (iv) to identify B cells that produce an antigen-specific antibody possessing at least one desired functional property; (vi) optionally pooling antigen-specific B cells obtained from different clonal B cell cultures (e.g., contained in different culture wells); (vii) staining the antigen-specific B cells obtained after step (iv) or after optional step (v) or optional step (vi) said with a label that facilitates positive or negative selection of the stained B cells; and (viii) sorting the stained antigen-specific B cells to isolate a single antigen-specific B cell. As disclosed infra, in some embodiments, the enrichment procedure may be effected 2 or 3 times.

The present invention also provides methods for cloning antigen-specific antibody variable sequences encoding the variable light chain region and/or the variable heavy chain region of the antibody expressed by the antigen-specific B cell identified using the methods outlined above. In one embodiment, the method for cloning includes steps (i)-(viii) above as well as (ix) placing the sorted B cells into a reverse transcription polymerase chain reaction (RT-PCR) reaction medium that facilitates the amplification of antigen-specific antibody variable sequences expressed by the sorted B cells; (x) sequencing the amplified nucleic acids encoding the antigen-specific antibody variable sequences; (xi) expressing the amplified nucleic acids or a variant thereof encoding the antigen-specific antibody variable sequences to produce antibody polypeptides; and (xii) determining which of the expressed antibody polypeptides bind to the antigen of interest.

In one embodiment, the host is a guinea pig, rabbit, mouse, rat, non-human primate or human. Preferably, the host is a rabbit. The B cells can be obtained from the host at about 20 to about 90 days after immunization, preferably the B cells are obtained from the host at about 50 to about 60 days after immunization.

In another embodiment, step (i) comprises harvesting B cells from at least one source selected from spleen, lymph node, bone marrow, and peripheral blood mononuclear cells from blood. In another embodiment, step (i) comprises harvesting B cells from more than one source selected from spleen, lymph node, bone marrow, peripheral blood mononuclear cells and blood and pooling said B cells from more than one source.

In one embodiment, the methods further comprise establishing a titer of antigen-specific antibodies (i.e., antibodies that specifically bind to the antigen) and/or neutralizing antibodies (i.e., antibodies that neutralize or inhibit binding of the antigen to a binding partner such as a receptor or ligand and/or to neutralize or inhibit at least one biological activity of the antigen) present in sera from the host.

In one embodiment, the enrichment step (ii) comprises affinity purification of antigen-specific B cells using an antigen directly or indirectly attached to a solid matrix, preferably magnetic beads, or support, preferably a column. In another embodiment, the antigen that is directly or indirectly attached to the solid matrix or support is biotinylated and attached to the matrix or support via streptavidin, avidin or neutravidin.

In a particular embodiment, the enrichment step (ii) comprises: (1) combining B cells with biotin-labeled antigen; (2) optionally washing the B cell/biotin-labeled antigen composition; (3) introducing streptavidin beads to the B cell/biotin-labeled antigen composition of (1) or (2); (4) passing the streptavidin beads/B cell/biotin-labeled antigen composition over a column; and (5) washing the column and eluting the bound B cells from the column, thereby obtaining an enriched antigen-specific B cell population. Alternatively, the enrichment step (ii) can comprise: (1) combining biotin-labeled antigen with streptavidin beads; (2) passing the biotin-labeled antigen/streptavidin bead composition over a column; (3) washing the column and eluting biotin-labeled antigen-coated beads from the column; (4) combining B cells with the coated beads; (5) passing the mixture of B cells and coated beads over the column; and (6) washing the column and eluting the bound B cells from the column, thereby obtaining an enriched antigen-specific B cell population. Either enrichment method, or a combination of both methods, can be repeated at least once thereby resulting in a further enriched antigen-specific B cell population.

In one embodiment, the enrichment step (ii) enriches the percentage of antigen-specific B cells by at least 2-fold, at least 5-fold, at least 10-fold, at least 50-fold, at least 100-fold, at least 1,000-fold or at least 10,000-fold. In another embodiment, the percentage of antigen-specific B cells in the enriched B cell population is at least 1%, 5%, or 10%.

In one embodiment, the enriched antigen-specific B cells are cultured in a medium comprising feeder cells, preferably irradiated EL4 cells. The medium can comprise activated T cell conditioned medium. Preferably, the enriched B cells are cultured in a medium comprising between about 1% and about 5% activated rabbit T cell conditioned medium (TSN). In exemplary embodiments, TSN may be produced by methods known in the art, such as those described in Seeber et al., “A Robust High Throughput Platform to Generate Functional Recombinant Monoclonal Antibodies Using Rabbit B Cells from Peripheral Blood,” PLoS ONE 9(2): e86184, and in publication no. EP 0488470 A1 (especially paragraph 0046), each of which is hereby incorporated by reference in its entirety.

The culturing can be effected for at least about 1-9 days, 2-8 days, 3-7 days, 4-6 days, or 5-7 days. Preferably, the culturing is effected for about 5-7 days.

In one embodiment, the enriched B cells are cultured in a multi-well plate with each well containing at least 1, at least 10, at least 25, at least 50, at least 100 or at least 200 enriched B cells. In another embodiment, each well contains about 50 to about 100 enriched B cells, about 25 to about 50 enriched B cells, or about 10 to about 25 enriched B cells. In a preferred embodiment, about 1 to about 200 of the enriched antigen-specific B cells are combined with irradiated EL4 cells and T cell supernatant (TSN) in each well of a multi-well plate.

In one embodiment, the antigen-recognition detection step (iv) comprises removing supernatant from the cultured enriched B cells and assaying said supernatant to identify the individual wells in the multi-well plate that contain antigen-reactive supernatants thereby detecting wells containing antigen-specific B cells. Preferably, the supernatant is evaluated by ELISA. In one embodiment, the antigen-reactive supernatants from the ELISA screen are transferred to another plate and freezing media is added to the original culture plate. In a particular embodiment, the supernatant is assayed for antigen-specific IgG production and total IgG production after culturing the enriched B cells for about 2 to about 7 days. The assay for total IgG production can be effected by (1) coating plates with an anti-species Fab, preferably an anti-rabbit Fab; (2) adding supernatant from cultured B cells to the plate; and (3) detecting the total IgG in the supernatant with an anti-species IgG, preferably an anti-rabbit IgG. Additionally, the assay for antigen-specific IgG production can be effected by (1) coating plates with unlabeled antigen or coating streptavidin plates with biotin-labeled antigen; (2) adding supernatant from cultured B cells to the plate; and (3) detecting the antigen-specific IgG in the supernatant with an anti-species IgG, preferably an anti-rabbit IgG. The ratio of antigen-specific wells to total IgG wells in the multi-well plate can correlate with B cell enrichment and clonality of the antibody secreting cell.

In one embodiment, the optional functional activity screening step (v) comprises assaying the antigen-reactive supernatants using an antigen-specific functional assay to identify wells that contain antigen-specific B cells that secrete antigen-specific antibodies having at least one desired functional property. In particular, the optional functional activity screening step (v) can comprise screening the antigen-specific B cells identified in step (iv) to identify B cells that produce an antigen-specific antibody that exhibits agonism or antagonism of antigen binding to a binding partner; induction or inhibition of the proliferation of a specific target cell type; induction or inhibition of lysis of a target cell; or induction or inhibition of a biological pathway involving the antigen. Exemplary functional activity screening steps include screening the antigen-specific antibody for induction or inhibition of the proliferation of T1165 cells; induction or inhibition of the proliferation of TF1 cells; induction or inhibition of cAMP production in SK-N-MC cells; or inhibition of PCSK9/LDLR interaction.

Generally, one or more freezing and storage steps can intervene one or more of the method steps.

In one embodiment, the staining step (vii) facilitates a negative antigen-specific B selection method. The negative antigen-specific B selection is effected by staining B cells with a first label that stains irradiated EL4 cells, preferably the first label is Thy1.2, and a second label that stains dead cells, preferably the second label is Propidium iodide (PI). Subsequent to staining for negative selection, the method further comprises sorting all viable, non-EL4 cells using flow cytometry, preferably performed using fluorescence-activated cell sorting (FACS) or immunomagnetic cell sorting (MACS).

In another embodiment, the staining step (vii) facilitates a positive antigen-specific B selection method. The positive antigen-specific B selection is effected by staining with a first label that stains species-specific B cells, preferably the first label is anti-rabbit IgG, and a second label that stains dead cells, preferably the second label is Propidium iodide (PI). Subsequent to staining for positive selection, the method further comprises sorting all viable, species-specific B cells using flow cytometry, preferably performed using FACS or MACS. The sorting step (viii) may include sorting the cells directly into RT-PCR reaction medium (for subsequent optional amplification and cloning), e.g., using FACS.

Additionally, the sorting step (viii) may further include optionally gating the sorted stained B cells. In a preferred embodiment, the optional gating step comprises selecting viable, non-EL4 cells that possess a distinct physical profile (FSC/SSC population). In another preferred embodiment, the optional gating step comprises selecting sorted viable, species-specific B cells that possess a distinct physical profile (FSC/SSC population), e.g., by drawing FSC/SSC physical gates that have no relation to autofluorescence levels. The optional gating step may further include sorting based on cell staining, which may include constructing a gate based on auto-fluorescence of unstained cells as a baseline for stained samples.

The sorting step can be performed using a single well sorting method or a pooled sorting method. For the pooled sorting method, different individual wells containing antigen-specific B cells secreting antigen-specific antibodies are combined prior to staining and cell sorting. In one embodiment, different individual wells containing antigen-specific B cells secreting antigen-specific antibodies having similar affinity and/or desired functional properties are combined prior to staining and sorting. Over 100 different ‘positive’ wells (i.e., identified as containing an antigen-specific B cell that produces a single antibody that binds to the desired antigen) from a multi-well plate can be combined for pooled sorting. Preferably, antigen-specific B cells from about 2 to about 10 different individual wells; about 10 to about 50 different individual wells; or about 50 to about 150 different individual wells are combined for pooled sorting.

In one embodiment, the methods further include an expression step (xi) that comprises expressing the sequenced and amplified nucleic acids encoding the antibody antigen-specific variable regions in a recombinant cell, such as a yeast, bacterium, plant, insect, amphibian or mammalian cell. Preferably, the recombinant cell is a diploid yeast, such as Pichia.

In another embodiment, the methods further include a determination step (xii) that comprises determining which of the expressed antibody polypeptides (e.g., resulting from recombinant expression of the sequenced and amplified nucleic acids encoding the antigen-specific variable sequences of the antibody isolated from the antigen-specific B cell) bind to the antigen of interest using radioimmunoassay (RIA), enzyme-linked immunoadsorbent assay (ELISA), immunoprecipitation, fluorescent immunoassays, western blot, surface plasmon resonance (BIAcore®) analysis or another antigen recognition assay. Preferably, the antigen binding specificity of the recombinant antibody polypeptide is determined using an ELISA assay.

In another embodiment, the invention further includes sorted populations of predominantly viable, non-EL4 cells produced according to the described B cell selection methods which sorted cell populations possess a distinct physical profile (FSC/SSC population), which preferably are obtained by flow cytometry using a negative antigen-specific B selection which preferably is effected by staining B cells with a first label that stains irradiated EL4 cells and a second label that stains dead cells, wherein the first label preferably is Thy1.2 and the second label preferably is Propidium iodide (PI). These sorted cells will preferably comprise B cells that secrete high affinity antibodies to desired antigens, especially antigens wherein antibodies specific thereto are potentially suitable for use in human therapy.

In another embodiment, the invention further includes sorted populations of viable, species-specific B cells produced according to the described B cell selection methods, which cell populations possess a distinct physical profile (FSC/SSC population), wherein the sorted populations of cells are preferably obtained by flow cytometry using a positive antigen-specific B selection, which is effected by staining with a first label that stains species-specific B cells and a second label that stains dead cells, wherein the first label preferably is anti-rabbit IgG and the second label preferably is Propidium iodide (PI). These sorted cells will also preferably comprise B cells that secrete high affinity antibodies to desired antigens, especially antigens wherein antibodies specific thereto are potentially suitable for use in human therapy.

### BRIEF DESCRIPTION OF THE FIGURES

FIG. 1 demonstrates that enrichment of harvested B cells improves the identification of antigen-specific B cells. 5 of 6 IgG-producing wells from an enriched B cell culture showed antigen-specificity, compared to 3 of 30 IgG-producing wells from a non-clonal B cell culture.

FIG. 2 shows a subpopulation of antigen-specific B cells having a larger, less granular phenotype (compared to the main cell population) collected using a final FSC/SSC gate during B cell sorting.

FIG. 3 shows antigen-specific B cells obtained via cell sorting exclusion of non-antigen specific B cells. The majority of cells stained were non-viable and/or irradiated feeder cells (Thy1.2+ and/or PI+). The viable, non-irradiated B cells (PI− and/or Thy1.2−) were selected and subject to a final FSC/SSC gate to obtain a subpopulation of cells with the desired physical phenotype, which were sorted into RT-PCR master mix.

FIG. 4 shows positive antigen-specific B cell selection during cell sorting. A small fraction of the total B cell population is IgG positive. The viable, IgG positive B cells (PI− and Rabbit IgG+) were selected and subject to a final FSC/SSC gate to obtain a subpopulation of cells with the desired physical phenotype, which were sorted into RT-PCR master mix.

FIG. 5 demonstrates that the FSC/SSC gated subpopulation of negative selected antigen-specific B cells have better than average amplification success. 26 of 88 FSC/SSC gated Thy1.2−/PI−B cells have the desired amplicon size, compared to 1 of the 88 Thy1.2−/PI−B cells (without the final FSC/SSC gate).

FIG. 6 depicts the binding affinity of two anti-PCSK9 antibodies (Ab1 and Ab2).

FIG. 7 depicts the functionality of two anti-PCSK9 antibodies (Ab1 and Ab2) in an LDL uptake assay.

FIG. 8 depicts the binding affinity of two anti-CGRP antibodies (Ab3 and Ab4).

FIG. 9 depicts the binding affinity of two anti-Target 1 antibodies (Ab5 and Ab6).

FIG. 10 depicts the binding affinity of two anti-NGF antibodies (Ab7 and Ab8).

FIG. 11 depicts the functionality of two anti-NGF antibodies (Ab7 and Ab7) in a TF1 proliferation assay.

FIG. 12 depicts the binding affinity of two anti-Target 2 antibodies (Ab9 and Ab10).

FIG. 13 depicts the functionality of the binding affinity of two anti-Target 2 antibodies (Ab9 and Ab10) in a HTRF assay.

FIG. 14 depicts the binding affinity of two anti-Target 3 antibodies (Ab11 and Ab12) determined by ELISA.

FIG. 15A-C provides a flow-chart illustrating two exemplary means for conducting the inventive antibody selection methodology.

### DETAILED DESCRIPTION OF THE INVENTION

The present invention provides methods of identifying antibody-secreting and antibody-forming cells, particularly rabbit antigen-specific B cells, and methods for cloning the antigen-specific sequences, e.g., V_{H} and/or V_{L} region of the antibodies produced by these cells. As described and exemplified infra, these methods contain a series of enrichment, culture, detection, screening, isolation, staining, sorting, amplification, sequencing, expression and determination steps that can be used in combination, sequentially, repetitively, or periodically. Preferably, these methods are used for identifying at least one antigen-specific B cell, which can be used to produce a monoclonal antibody that is specific to a desired antigen, or a nucleic acid sequence corresponding to such an antibody or a variant thereof.

In the methods of the present invention, an antibody is selected after an enrichment step, a culture step that results in a clonal population of antigen-specific B cells, a detection step that results in identifying antigen-specific B cells using antigen-recognition assay, an optional screening test to identify antigen-specific B cells that produce an antigen-specific antibody with a desired functional property, a staining step for positive or negative selection of the stained cells, and a sorting step to obtain a single antigen-specific B cell.

The methods can further comprise a step of sequencing a selected antibody or portions thereof from one or more isolated, antigen-specific cells. Any method known in the art for sequencing can be employed and can include sequencing the heavy chain, light chain, variable region(s), and/or complementarity determining region(s) (CDR). Preferably, the methods include an enrichment step, a culture step and a sequencing step.

In one embodiment, the present invention provides a method for identifying an antigen-specific B cell (i.e., expresses an antigen-specific antibody) comprising:
(i) obtaining B cells from a host that has been immunized or exposed naturally to an antigen of interest;
(ii) enriching a fraction of said B cells to obtain an enriched population of antigen-specific B cells, which contains a greater percentage of B cells that produce an antibody that binds to the antigen of interest relative to the B cell fraction prior to enrichment;
(iii) separately culturing one or more fractions from said enriched antigen-specific B cell population under culture conditions that favor the formation of a clonal B cell population that produces a single antibody that binds to the antigen of interest;
(iv) detecting the clonal B cell population that produces a single antibody that binds to the antigen of interest, thereby identifying one or more antigen-specific B cells;
(v) optionally screening the clonal antigen-specific B cell population identified in step (iv) to identify B cells that produce an antigen-specific antibody possessing at least one desired functional property;
(vi) optionally pooling antigen-specific B cells obtained from different clonal B cell cultures;
(vii) staining the antigen-specific B cells obtained after step (iv) or after optional step (v) or optional step (vi) said with a label that facilitates positive or negative selection of the stained B cells; and
(viii) sorting the stained antigen-specific B cells and optionally gating the sorted stained B cells to isolate a single antigen-specific B cell.

Moreover, the method further comprise cloning the antigen-specific antibody variable sequences encoding the variable light chain region and/or the variable heavy chain region by:
(ix) placing the sorted B cells into a reverse transcription polymerase chain reaction (RT-PCR) reaction medium that facilitates the amplification of antigen-specific antibody variable sequences expressed by the sorted B cells;
(x) sequencing the amplified nucleic acids encoding the antigen-specific antibody variable sequences;
(xi) expressing the amplified nucleic acids or a variant thereof encoding the antigen-specific antibody variable sequences to produce antibody polypeptides; and
(xii) determining which of the expressed antibody polypeptides bind to the antigen of interest.

The inventive B cell selection protocol disclosed herein has a number of intrinsic advantages versus other methods for obtaining antibody-secreting B cells and monoclonal antibodies specific to desired target antigens. These advantages include, but are not restricted to, the following:

First, it has been found that when these selection procedures are utilized with a desired antigen, such as PCSK9, CGRP, Target 1, NGF or Target 2, the methods reproducibly result in antigen-specific B cells capable of generating what appears to be a substantially comprehensive complement of antibodies, i.e., antibodies that bind to the various different epitopes of the antigen. Without being bound by theory, it is hypothesized that the comprehensive complement is attributable to the antigen enrichment step that is performed prior to initial B cell recovery. Moreover, this advantage allows for the isolation and selection of antibodies with different properties as these properties may vary depending on the epitopic specificity of the particular antibody.

Second, it has been found that the inventive B cell selection protocol reproducibly yields a clonal B cell culture containing a single B cell, or its progeny, secreting a single monoclonal antibody that generally binds to the desired antigen with a relatively high binding affinity, i.e. close to picomolar antigen binding affinities. By contrast, prior antibody selection methods tend to yield relatively few high affinity antibodies and therefore require extensive screening procedures to isolate an antibody with therapeutic potential. Without being bound by theory, it is hypothesized that the inventive protocol results in both in vivo B cell immunization of the host (primary immunization) followed by a second in vitro B cell stimulation (secondary antigen priming step) that may enhance the ability and propensity of the recovered clonal B cells to secrete a single high affinity monoclonal antibody specific to the antigen target.

Third, it has been observed (as shown herein with PSCK9, CGRP, Target 1, NGF and Target 2 specific B cells) that the inventive B cell selection protocol reproducibly yields enriched B cells producing IgGs that are, on average of high quality, i.e., highly selective (antigen specific) to the desired target and/or exhibiting desired functional properties. In part based thereon, antigen-enriched B cells recovered by the inventive methods are believed to contain B cells capable of yielding the desired full complement of epitopic specificities as discussed above.

Fourth, it has been observed that the inventive B cell selection protocols, even when used with small antigens, i.e., peptides of 100 amino acids or less, e.g., 5-50 amino acids long, reproducibly give rise to a clonal B cell culture that secretes a single high affinity antibody to the small antigen, e.g., a peptide. This is highly surprising as it is generally quite difficult, labor intensive, and sometimes not even feasible to produce high affinity antibodies to small peptides. Accordingly, the invention can be used to produce therapeutic antibodies to desired peptide targets, e.g., viral, bacterial or autoantigen peptides, thereby allowing for the production of monoclonal antibodies with very discrete binding properties or even the production of a cocktail of monoclonal antibodies to different peptide targets, e.g., different viral strains. This advantage may especially be useful in the context of the production of a therapeutic or prophylactic vaccine having a desired valency, such as an HPV vaccine that induces protective immunity to different HPV strains.

Fifth, the inventive B cell selection protocol, particularly when used with B cells derived from rabbits, tends to reproducibly yield antigen-specific antibody sequences that are very similar to endogenous human immunoglobulins (around 90% similar at the amino acid level) and that contain CDRs that possess a length very analogous to human immunoglobulins and therefore require little or no sequence modification (typically at most only a few CDR and/or framework residues may be modified in the parent antibody sequence) in order to eliminate potential immunogenicity concerns. In particular, preferably the recombinant antibody will contain only the host (rabbit) CDR1 and CDR2 residues required for antigen recognition and the entire CDR3. Thereby, the high antigen binding affinity of the recovered antibody sequences produced according to the inventive B cell and antibody selection protocol remains intact or substantially intact even with humanization.

In sum, the inventive method can be used to produce antibodies exhibiting higher binding affinities to more distinct epitopes by the use of a more efficient protocol than was previously known.

Obtaining Antibody-Secreting Cells

The methods disclosed herein include a step of obtaining an immune cell-containing cell population from an immunized host. Methods of obtaining an immune cell-containing cell population from an immunized host are known in the art and generally include inducing an immune response in a host and harvesting cells from the host to obtain one or more cell populations. The response can be elicited by immunizing the host against a desired antigen. Alternatively, the host used as a source of such immune cells can be naturally exposed to the desired antigen such as an individual who has been infected with a particular pathogen such as a bacterium or virus or alternatively has mounted a specific antibody response to a cancer that the individual is afflicted with.

Host animals are well-known in the art and include, but are not limited to, guinea pig, rabbit, mouse, rat, non-human primate, human, as well as other mammals and rodents, chicken, cow, pig, goat, and sheep. Preferably the host is a mammal; more preferably a rabbit, mouse, rat, or human; most preferably, a rabbit. When exposed to an antigen, the host produces antibodies as part of the native immune response to the antigen. As mentioned, the immune response can occur naturally, as a result of disease, or it can be induced by immunization with the antigen. Immunization can be performed by any method known in the art, such as, by one or more injections of the antigen with or without an agent to enhance immune response, such as complete or incomplete Freund's adjuvant. As an alternative to immunizing a host animal in vivo, the method can comprise immunizing a host cell culture in vitro or DNA immunization.

After allowing time for the immune response (e.g., as measured by serum antibody detection), host animal cells are harvested to obtain one or more immune cell-containing cell populations. A harvested cell population is preferably from at least one of the spleen, lymph nodes, bone marrow, blood and/or peripheral blood mononuclear cells (PBMCs). The cells can be harvested from more than one source and pooled. Certain sources may be preferred for certain antigens. For example, the spleen, lymph nodes, and whole blood are preferred for PCSK9, CGRP, Target 1, NGF and Target 2. The titer of antigen-specific and/or neutralizing antibodies present in the sera of the host animal can then be determined.

The cell population is harvested about 20 to about 90 days or increments therein after immunization, preferably about 50 to about 60 days. A harvested cell population and/or a single cell suspension therefrom can be enriched, screened, and/or cultured for antibody selection. The frequency of antigen-specific cells within a harvested cell population is usually about 1% to about 5%, or increments therein.

Throughout this application, the term “increment” is used to define a numerical value in varying degrees of precision, e.g., to the nearest 10, 1, 0.1, 0.01, etc. The increment can be rounded to any measurable degree of precision, and the increment need not be rounded to the same degree of precision on both sides of a range. For example, the range 1 to 100 or increments therein includes ranges such as 20 to 80, 5 to 50, and 0.4 to 98. When a range is open-ended, e.g., a range of less than 100, increments therein means increments between 100 and the measurable limit. For example, less than 100 or increments therein means 0 to 100 or increments therein unless the feature, e.g., temperature, is not limited by 0.

Enrichment of Antibody-Secreting Cells

The present invention provides an improvement to existing methods of isolating a single antibody-producing B cell. In particular, the methods include an enrichment step (ii) which involves enriching B cells obtained from a host thereby resulting in obtaining an enriched population of B cells. As a result of the enrichment step, subsequent culturing steps require fewer cells, e.g., individual wells in multi-well tissue culture plates can be seeded at lower B cell culture concentrations and still achieve desired success rates. For example, as few as about 10 or about 25 of the enriched B cells can be subsequently cultured in each well of a multi-well plate and still yield antigen-specific antibodies.

In contrast to prior techniques, where antibodies are produced from a cell population with a low frequency of antigen-specific cells, the present invention allows antibody selection from among a high frequency of antigen-specific cells. Because an enrichment step is used prior to antibody selection, the majority of the cells, preferably virtually all of the cells, used for antibody production are antigen-specific. By producing antibodies from a population of cells with an increased frequency of antigen specificity, the quantity and variety of antibodies are increased.

The enriched population of B cells contains a greater percentage of antigen-specific B cells, i.e., cells that produce an antibody that binds to the antigen of interest, relative to the B cell sample prior to enrichment. In one embodiment, the percentage of antigen-specific B cells in the enriched B cell population is at least 1%, 5% or 10%.

The enrichment step precedes any selection step(s), e.g., selecting a particular B cell from a cell population and/or selecting an antibody produced by a particular cell. After culturing the enriched B cell population under conditions that favor the formation of a clonal B cell population, enrichment results in obtaining a clonal population of B cells that produces a single monoclonal antibody specific to said antigen.

Throughout this application, a “clonal population of B cells” refers to a population of B cells that only secrete a single antibody specific to a desired antigen. That is to say that these cells produce only one type of monoclonal antibody specific to the desired antigen.

In the present application, “enriching” a cell population cells means increasing the frequency of desired cells, typically antigen-specific B cells, contained in a mixed cell population, e.g., a B cell-containing isolate derived from a host that is immunized against a desired antigen. Thus, an enriched cell population encompasses a cell population having a higher frequency and/or higher percentage of antigen-specific cells as a result of an enrichment step, but this population of cells may contain and produce different antibodies.

The general term “cell population” encompasses pre- and a post-enrichment cell populations, keeping in mind that when multiple enrichment steps are performed, a cell population can be both pre- and post-enrichment. For example, the enrichment step can be performed as one, two, three, or more steps. In one embodiment, the present invention provides a method that includes multiple enrichment steps, such as:
(a) obtaining B cells from a host that has been immunized or exposed naturally to an antigen of interest, and creating at least one single cell suspension from the harvested cell population;
(b) enriching a fraction of said B cell single cell suspension to obtain a first enriched population of antigen-specific B cells, which contains a greater percentage of B cells that produce an antibody that binds to the antigen of interest relative to the B cell fraction prior to enrichment;
(c) enriching the first enriched antigen-specific B cell population to form a second enriched antigen-specific B cell population, which contains a greater percentage of antigen-specific B cells relative to the first enriched antigen-specific B cell population;
(d) enriching the second enriched antigen-specific B cell population to form a third enriched antigen-specific B cell population, which contains a greater percentage of antigen-specific B cells relative to the second enriched antigen-specific B cell population;
(e) culturing the third enriched antigen-specific B cell population to generate a clonal B cell population that produces a single antibody that binds to the antigen of interest; and
(f) selecting an antibody produced by an antigen-specific cell isolated from the third enriched cell population.

Each cell population may be used directly in the next step, or it can be partially or wholly frozen (e.g., at −70° C. or −80° C. or in liquid nitrogen) for long- or short-term storage or for later steps, e.g., detection, isolation, staining and sorting. Also, cells from a cell population can be individually suspended to yield single cell suspensions. The single cell suspension can be enriched, such that a single cell suspension serves as the pre-enrichment cell population. Then, one or more antigen-specific single cell suspensions together form the enriched cell population; the antigen-specific single cell suspensions can be grouped together, e.g., re-plated for further analysis and/or antibody production.

As mentioned, the enriched B cell population used in the inventive process can also be further enriched, screened, and/or cultured for antibody selection according to the steps described herein, which can be repeated or performed in a different order. In a preferred embodiment, at least one cell of an enriched, preferably clonal, antigen-specific cell population is isolated, cultured, and used for antibody selection. Thus, in one embodiment, the present invention provides a method comprising:
a. harvesting a cell population from an immunized host to obtain a harvested cell population;
b. creating at least one single cell suspension from a harvested cell population;
c. enriching at least one single cell suspension, preferably by chromatography, to form a first enriched cell population;
d. enriching the first enriched cell population, preferably by ELISA assay, to form a second enriched cell population which preferably is clonal, i.e., it contains only a single type of antigen-specific B cell;
e. enriching the second enriched cell population, preferably by ELISA assay, to form a third enriched cell population containing a single or a few number of B cells that produce an antibody specific to a desired antigen;
f. culturing the third enriched cell population to generate a clonal cell population that produces a single antibody that binds to the antigen of interest; and
g. selecting an antibody produced by an antigen-specific cell isolated from the third enriched cell population.

In one embodiment, a single B cell is isolated from an enriched cell population before confirming whether the single B cell secretes an antibody with antigen-specificity and/or a desired property. In another embodiment, the enriched B cells are cultured in a multi-well plate with each individual well containing about 1 to about 200 enriched B cells enriched B cells. For example, each individual well of the multi-well plate contains at least 1, at least 10, at least 25, at least 50, at least 100 or at least 200 enriched B cells. Preferably, about 50 to about 100 enriched B cells, about 25 to about 50 enriched B cells, or about 10 to about 25 enriched B cells are seeded per well.

In one embodiment, the present invention provides a method of enriching a cell population to yield an enriched cell population having an antigen-specific cell frequency that is about 50% to about 100%, or increments therein. Preferably, the enriched cell population has an antigen-specific cell frequency greater than or equal to about 50%, 60%, 70%, 75%, 80%, 90%, 95%, 99%, or 100%.

In another embodiment, the present invention provides a method of enriching a cell population whereby the frequency of antigen-specific cells is increased by at least about 2-fold, 5-fold, 10-fold, 20-fold, 50-fold, 100-fold, 1000-fold or 10,000-fold or increments therein.

Antigen-specificity can be measured with respect to any antigen. The antigen can be any substance to which an antibody can bind including, but not limited to, peptides, proteins or fragments thereof; carbohydrates; organic and inorganic molecules; receptors produced by animal cells, bacterial cells, and viruses; enzymes; agonists and antagonists of biological pathways; hormones; and cytokines. Exemplary antigens include, but are not limited to, IL-2, IL-4, IL-6, IL-10, IL-12, IL-13, IL-18, IFN-α, IFN-γ, Angiotensin II, BAFF, CGRP, CXCL13, IP-10, PCSK9, NGF, Nav1.7, VEGF, EPO, EGF, and HRG. Preferred antigens include CGRP, PCSK9, Nav1.7, NGF, Angiotensin II, IL-6, IL-13, TNF-α and VEGF-α.

In a method utilizing more than one enrichment step, the antigen used in each enrichment step can be the same as or different from the antigen used in another enrichment step. Multiple enrichment steps with the same antigen may yield a large and/or diverse population of antigen-specific cells, whereas multiple enrichment steps with different antigens may yield an enriched cell population with cross-specificity to the different antigens.

Enriching a cell population can be performed by any cell-selection means known in the art for isolating antigen-specific cells. Exemplary antigen binding assays include radioactive assays and non-radioactive assays non-radioactive assays based on optical methods, e.g., fluorescence, phosphorescence, chemoluminescence, electrochemoluminescence, fluorescence polarization, fluorescence resonance energy transfer or surface plasmon resonance. In one embodiment, the detection of antigen-recognition comprises radioimmunoassay (RIA), enzyme-linked immunoadsorbent assay (ELISA), immunoprecipitation, fluorescent immunoassays, western blot, surface plasmon resonance (ProteOn or BIAcore®) analysis or another antigen binding assay. Preferably, antigen-recognition is performed using ELISA.

A cell population can be enriched by chromatographic techniques, e.g., affinity purification. For example, antigen-specific B cells can be purified using an antigen directly or indirectly attached to a solid matrix (e.g., magnetic heads, such as Miltenyi MACS® MicroBeads, or non-magnetic beads, such as agarose or polyacrylamide beads) or support (e.g., magnetic columns, such Miltenyi MS columns (Miltenyi Biotech), or non-magnetic columns, such as spin columns and gravity flow columns).

In one embodiment, a single cell suspension from a harvested cell population is enriched, preferably by using Miltenyi beads. For example, cells in a single-cell suspension can be magnetically labeled with MACS® MicroBeads, and the sample can be applied to a MACS® Column placed in a MACS® Separator. The unlabeled cells pass through while the magnetically labeled cells are retained within the column. The flow-through can be collected as the unlabeled cell fraction. After a short washing step, the column can be removed from the separator, and the magnetically labeled cells can be eluted from the column.

From the harvested cell population having a frequency of antigen-specific cells of about 1% to about 5%, an enriched cell population is thus derived having a frequency of antigen-specific cells approaching 100%.

The antigen of interest can be directly or indirectly attached to the solid matrix or support. For example, the antigen can be biotinylated and attached to the matrix or support via streptavidin, avidin or neutravidin. In one embodiment, the enrichment step (ii) comprises affinity purification of antigen-specific B cells using an antigen directly or indirectly attached to a solid matrix or support, such as magnetic beads or a column. Preferably, the antigen is biotinylated and attached to the matrix or support via streptavidin, avidin, or neutravidin.

In one embodiment, the enrichment step comprises: (1) combining B cells with biotin-labeled antigen; (2) optionally washing the B cell/biotin-labeled antigen composition; (3) introducing streptavidin beads to the B cell/biotin-labeled antigen composition of (1) or (2); (4) passing the streptavidin beads/B cell/biotin-labeled antigen composition over a column; and (5) washing the column and eluting the bound B cells from the column, thereby obtaining an enriched antigen-specific B cell population. Alternatively, in another embodiment, the enrichment step comprises: (1) combining biotin-labeled antigen with streptavidin beads; (2) passing the biotin-labeled antigen/streptavidin bead composition over a column; (3) washing the column and eluting biotin-labeled antigen-coated beads from the column; (4) combining B cells with the coated beads; (5) passing the mixture of B cells and coated beads over the column; and (6) washing the column and eluting the bound B cells from the column, thereby obtaining an enriched antigen-specific B cell population. These enrichment methods, or a combination thereof, can be used and optionally repeated at least once resulting in a further enriched antigen-specific B cell population.

A cell population can also be enriched by performing any antigen-specificity assay technique known in the art. For example, a halo assay, which comprises contacting the cells with antigen-loaded beads and labeled, e.g., a fluorophore, anti-host antibody specific to the host used to harvest the B cells, may be used. However, in a preferred embodiment, flow cytometry is used to enrich the cell population. As discussed below, antigen-specific B selection can be isolated by staining and sorting. Briefly, fluorescence-activated cell sorting (FACS) or immunomagnetic cell sorting (MACS) can be used to select antigen-specific B cells based on desired properties, e.g., viability, IgG expression and/or size.

In one embodiment, at least one assay enrichment step is performed on at least one single cell suspension. In another embodiment, the method of enriching a cell population includes at least one chromatographic enrichment step and at least one assay enrichment step.

Methods of “enriching” a cell population by size or density are known in the art. See, e.g., U.S. Pat. No. 5,627,052. These steps can be used in the present method in addition to enriching the cell population by antigen-specificity.

The cell populations of the present invention contain at least one cell capable of recognizing an antigen. Antigen-recognizing cells include, but are not limited to, B cells, plasma cells, and progeny thereof. In one embodiment, the present invention provides a clonal cell population containing a single type of antigen-specific B cell, i.e., the B cell population produces a single monoclonal antibody that specifically binds to a desired antigen.

It is believed that the clonal antigen-specific population of B cells consists predominantly of antigen-specific, antibody-secreting cells, which are obtained by the novel culture and selection protocol provided herein. Accordingly, the present invention also provides methods for obtaining an enriched cell population containing at least one antigen-specific, antibody-secreting cell. In one embodiment, the present invention provides an enriched cell population containing about 50% to about 100%, or increments therein, or greater than or equal to about 60%, 70%, 80%, 90%, or 100% of antigen-specific, antibody-secreting cells. Preferably, the enriched cell population comprises no more than about 10,000 antigen-specific, antibody-secreting cells, more preferably about 50-10,000, about 50-5,000, about 50-1,000, about 50-500, about 50-250 antigen-specific, antibody-secreting cells, or increments therein.

The enriched antigen-specific B cells are subsequently cultured, detected by antigen-recognition assays, optionally screened for functional activity, isolated, stained and sorted prior to optional steps of amplifying the nucleic acids encoding the antigen-specific antibody variable sequences (e.g., V_{H} and V_{L} chain), sequencing of the amplified nucleic acids, expression of the nucleic acids to produce the corresponding antibody polypeptides and determination of the resulting antibody's antigen-recognition.

Enrichment of a cell population is used in a method comprising antibody production and/or selection in order to clone antibody sequences that express an antigen-specific variable heavy region and/or variable light region. Thus, the present invention provides a method comprising enriching a cell population before selecting an antibody. The method can include the steps of: preparing a cell population comprising at least one antigen-specific cell, enriching the cell population by isolating at least one antigen-specific cell to form an enriched cell population, and inducing antibody production from at least one antigen-specific cell. In a preferred embodiment, the enriched cell population contains more than one antigen-specific cell.

Culturing Enriched Antibody-Secreting Cell Populations

The methods also include a culturing step, in which the cell populations can be cultured with suitable medium (e.g., an activated T cell conditioned medium, particularly 1-5% activated rabbit T cell conditioned medium) on a feeder layer, preferably under conditions that favor the survival of a single proliferating antibody-secreting cell per culture well. The feeder layer, generally comprised of irradiated cell matter, e.g., EL4B cells, does not constitute part of the cell population. The cells are cultured in a suitable media under suitable conditions for a time sufficient for antibody production, for example about 1 day to about 2 weeks, about 1 day to about 10 days, at least about 3 days, about 3 to about 5 days, about 5 days to about 7 days, at least about 7 days, or other increments therein. Preferably, a single antibody-producing cell and progeny thereof survives in each well, thereby providing a clonal population of antigen-specific B cells in each well.

One or more fractions of the enriched cell population from step (ii) is/are separately cultured under conditions that favor the formation of a clonal cell population, i.e., produces a single antibody that binds to the antigen of interest. In one embodiment, more than one fraction of the enriched cell population is separately cultured simultaneously with another fraction from the same enriched cell population.

In one embodiment, the antigen-specific B cells of the enriched B cell population obtained in step (ii) are cultured under conditions that yield a clonal antigen-specific B cell population before isolating an antibody producing cell therefrom and/or producing an antibody using said B cell, or a nucleic acid sequence corresponding to such an antibody.

Cells from the enriched population can be combined and cultured with feeder cells. In one embodiment, the enriched cells are cultured under these conditions for at least about 1-9 days, about 2-8 days, about 3-7 days, about 4-6 days, or, preferably, about 5-7 days. In one embodiment, B cells from enriched antigen-specific B cell population are cultured in medium containing activated T cell conditioned medium with feeder cells, preferably irradiated EL4 cells (e.g., EB4 cell subline EB4.B5). In a preferred embodiment, the enriched B cells are cultured in a medium comprising between about 1% and about 5% activated rabbit T cell conditioned medium.

In one embodiment, an enriched cell population, such as an antigen-specific single cell suspension from a harvested cell population, is plated at various cell densities (e.g., 10, 25, 50, 100, 250, 500, or other increments between 1 and 1000 cells per well) and cultured in a multi-well plate. For example, the enriched B cells can be cultured in a multi-well plate with each well containing at least 1, at least 10, at least 25, at least 50, at least 100 or at least 200 enriched B cells. Preferably, each well contains about 10 to about 100 enriched B cells, about 25 to about 50 enriched B cells, or about 10 to about 25 enriched B cells. As a result of the enrichment step, subsequent culturing steps require fewer cells, e.g., individual wells in multi-well tissue culture plates can be seeded at lower B cell culture concentrations and still achieve desired success rates.

At this stage, the immunoglobulin G (IgG) produced by the clonal population is highly correlative with antigen specificity. In a preferred embodiment, the IgGs exhibit a correlation with antigen specificity that is greater than about 50%, more preferably greater than 70%, 85%, 90%, 95%, 99%, or increments therein. The correlations were demonstrated by setting up B cell cultures under limiting conditions to establish single antigen-specific antibody products per well. Antigen-specific versus general IgG synthesis was compared. Three populations were observed: IgG that recognized a single format of antigen (biotinylated and direct coating), detectable IgG and antigen recognition irrespective of immobilization, and IgG production alone. IgG production was highly correlated with antigen-specificity.

Screening Antibody-Secreting Cells for Antigen-Recognition and Functional Activity

In addition to the enrichment step, the method for antibody selection also include one or more steps of screening a cell population for antigen recognition and optionally antibody functionality. For example, the desired antibodies may have specific structural features, such as binding to a particular epitope or mimicry of a particular structure; antagonist or agonist activity; or neutralizing activity, e.g., inhibiting binding between the antigen and a ligand. In one embodiment, the antibody functionality screen is ligand-dependent.

In one embodiment, the enriched, preferably clonal, antigen-specific B cell population from which a supernatant described above is optionally screened in order to detect the presence of the desired secreted monoclonal antibody is used for the isolation of a few B cells, preferably a single B cell, which is then tested in an appropriate assay in order to confirm the presence of a single antibody-producing B cell in the clonal B cell population. In one embodiment about 1 to about 20 cells are isolated from the clonal B cell population, preferably less than about 15, 12, 10, 5, or 3 cells, or increments therein, most preferably a single cell. The screen is preferably effected by an antigen-specificity assay, especially an ELISA assay (e.g., selective antigen immobilization using a biotinylated antigen capture by streptavidin coated plate as described above).

The antibody-containing supernatant can also be screened for at least one of: antigen binding affinity; agonism or antagonism of antigen-ligand binding, induction or inhibition of the proliferation of a specific target cell type; induction or inhibition of lysis of a target cell, and induction or inhibition of a biological pathway involving the antigen. Suitable screening steps include, but are not limited to, assay methods that detect: whether the antibody produced by the identified antigen-specific B cell produces an antibody possessing a minimal antigen binding affinity, whether the antibody agonizes or antagonizes the binding of a desired antigen to a ligand; whether the antibody induces or inhibits the proliferation of a specific cell type; whether the antibody induces or elicits a cytolytic reaction against target cells; whether the antibody binds to a specific epitope; and whether the antibody modulates (inhibits or agonizes) a specific biological pathway or pathways involving the antigen.

Screening for antibody functionality includes, but is not limited to, an in vitro protein-protein interaction assay that recreates the natural interaction of the antigen ligand with recombinant receptor protein; and a cell-based response that is ligand dependent and easily monitored (e.g., proliferation response). In one embodiment, antibody functionality includes T1165 cell proliferation, TF1 cell proliferation, cAMP production in SK-N-MC cells or PCSK9/LDLR inhibition.

Generally, a supernatant containing the antibodies is collected, which can be can be enriched, screened, and/or cultured for antibody selection according to the steps described above. In one embodiment, the supernatant is enriched (preferably by an antigen-specificity assay, especially an ELISA assay) and/or screened for antibody functionality.

In one embodiment, the method for antibody selection includes a step of screening the cell population for antibody functionality by measuring the percent (%) inhibition. Upon obtaining a recombinant antibody expressed from amplified and sequenced nucleic acids encoding the antigen-specific variable regions of an antibody produced from an enriched B cells with antigen specificity, the inhibitory concentration (IC₅₀) may be determined. In one embodiment, at least one of the isolated, antigen-specific cells produces an antibody having an IC₅₀ of less than about 100, 50, 30, 25, 10 μg/mL, or increments therein.

In another embodiment, the method for antibody selection includes a step of screening the cell population for antibody binding strength. Antibody binding strength can be measured by any method known in the art (e.g., surface plasmon resonance (Biacore®)). At least one of the isolated, antigen-specific cells may produce an antibody having a high antigen affinity, e.g., a dissociation constant (K_{D}) of less than about 5×10⁻¹⁰ M⁻¹, preferably about 1×10⁻¹³ to 5×10⁻¹⁰, 1×10⁻¹² to 1×10⁻¹⁰, 1×10⁻¹² to 7.5×10⁻¹¹, 1×10⁻¹¹ to 2×10⁻¹¹, about 1.5×10⁻¹¹ or less, or increments therein. In this embodiment, the antibodies are said to be affinity mature. For example, the affinity of the antibodies is comparable to or higher than the affinity of any one of Panorex® (edrecolomab), Rituxan® (rituximab), Herceptin® (traztuzumab), Mylotarg® (gentuzumab), Campath® (alemtuzumab), Zevalin™ (ibritumomab), Lrbitux™ (cetuximab), Avastin™ (bevicizumab), Raptiva™ (efalizumab), Remicade® (infliximab), Humira™ (adalimumab), and Xolair™ (omalizumab). The affinity of an antibody can also be increased by known affinity maturation techniques. In one embodiment, at least one cell population is screened for at least one of, preferably both, antibody functionality and antibody binding strength.

In addition to the enrichment step, the method for antibody selection includes one or more steps of screening the cell population for antibody sequence homology, especially human homology. In one embodiment, at least one of the isolated, antigen-specific cells produces an antibody that has a homology to a human antibody of about 50% to about 100%, or increments therein, or greater than about 60%, 70%, 80%, 85%, 90%, or 95% homologous. The antibodies can be humanized to increase the homology to a human sequence by techniques known in the art such as CDR grafting or selectivity determining residue grafting (SDR).

In another embodiment, the present invention also provides the antibodies themselves according to any of the embodiments described above in terms of IC₅₀, K_{D}, and/or homology.

Isolation of Antibody-Secreting Cells: Staining and Sorting

In addition to the enrichment step, the method for antibody selection also includes one or more steps of staining and sorting antibody-secreting cells to isolate a single antibody-producing cell. In particular, single antigen-specific cells in the clonal population can be isolated by staining the cell population to identify antigen-specific cells having a specific phenotype, e.g., viability, surface marker expression, etc., using one or more labels that facilitate positive or negative selection of the stained cells.

In one embodiment, antigen-specific B cells from an enriched clonal population are stained for subsequent sorting. Exemplary labels for staining antigen-specific B cells include fluorescent and non-fluorescent reagents that bind to the gamma, kappa or lambda surface chain; CD19; CD27; IgG; IgD; Ia; Fc receptors; or desired antigen as well as reagents that selectively stain dead cells (e.g., PI or 7-AAD) or live cells (e.g., calcein dyes). For increasing the specificity of sorting, multiple labels that target the desired antigen-specific cells can be used. When using multiple fluorescent labels, distinct excitation/emission wavelengths are selected such that by using one or two lasers, cells labeled with two, three, four or more colors can be sorted.

Typically, detection reagents are labeled or are amenable to labeling indirectly via a secondary detection reagent that binds to the detection reagent. Such labeling can be fluorescence, isotopic, magnetic, and paramagnetic among others. For example, a fluorescent label or dye can be used to identify single cells with certain physical characteristics. Examples of fluorescent labels include PI, FITC, PE, PC5 (PE-Cy5), ECD (PE-Texas Red), and Cy-Chrome (R-PE) which can be detected using 630, 525 nm, 575 nm, 675 nm, 610 nm, and 650 nm band pass filters. A fluorescent label can be conjugated to a monoclonal antibody that specifically identifies a particular cell type based on the individual antigenic surface markers of the cell. In a mixed population of cells, different fluorescent labels can be used to distinguish separate subpopulations. If more than one detection reagent is used, then the different detection reagents are differentially labeled (e.g., using different fluorophores).

In one embodiment, the antigen-specific cells are stained with a label that facilitates positive or negative selection. The staining step employs at least two labels with different fluorochromes. Exemplary methods for staining cells using immunofluorescence are provided in Radbruch, A., Flow Cytometry and Cell Sorting (Springer, 2^{nd} Ed. 2010), Chapter 3. For example, for positively selecting viable B cells expressing antibodies with a specificity to an antigen of interest, the B cells are labeled with an anti-IgG antibody coupled to a fluorochrome and a viability dye. Preferably, the positive staining of enriched antigen-specific B cells comprises staining the cells with a first label that stains IgG-producing cells (e.g., FITC-anti-Rabbit Fc) and a second label that stains dead cells (e.g., PI or 7-AAD). In a specific embodiment, antigen-specific B cells stained with FITC-anti-IgG and PI were excited by the 488 nm spectral line of an argon laser. Alternatively, for negatively selecting viable antigen-specific B cells, the cells are labeled with an fluorochrome-coupled antibody that binds to other cells, e.g., feeder cells, that may be present in the sample and a viability dye. Preferably, the negative staining of the enriched antigen-specific B cells comprises staining the cells with a first label that stains feeder cells (e.g., Thy1.2) and a second label that stains dead cells (e.g., PI or 7-AAD). In a specific embodiment, antigen-specific B cells stained with PE-anti-Thy1.2 and PI were excited by the 488 nm spectral line of an argon laser. Green (FITC) and red (PE and PI) fluorescence was collected using 525 nm, 575 nm and 630 nm long pass band filters, respectively.

Moreover, in some embodiments, several individual wells containing antigen-specific cells are combined or ‘pooled’ prior to staining and sorting (also called a ‘pooled cell sort’). Preferably, individual wells containing antigen-specific B cells secreting antibodies that have similar properties, e.g., binding affinity or functionality, are combined prior to staining and sorting. Any number of positively identified wells, i.e., containing antigen-specific cells from a clonal population, may be combined prior to the staining and sorting step. In one embodiment, about 2 to about 200, about 10 to about 100, about 25 to about 75 wells are combined prior to staining and sorting. Preferably, about 2 to about 10, about 10 to about 50 wells, or about 50 to about 150 wells are combined prior to staining and sorting. Pooled cell sorting increases the throughput capacity and minimizes benchwork. Antibodies resulting from unique sequences that are identified from a pooled cell sort and carried forward through the optional cloning process can be traced back to the pool, but not a specific well of origin.

Alternatively, individual wells containing antigen-specific cells may be separately stained and sorted (also called a ‘single well sort’). Single well sorting has limited throughput capacity, but antibodies resulting from cloning unique sequences can be directly traced to their well of origin. Additionally, single well sorting provides more direct correlation with primary results from the original screen.

In addition to the enrichment, culturing and staining steps, the methods also include a sorting step in which the stained antigen-specific cells are sorted into populations and subpopulations based on the presence of absence of labels used to stain cells of a certain desired phenotype. Sorting allows one to capture and collect cells of interest for further analysis. Once collected, the cells can be analyzed microscopically, biochemically, or functionally. The stained cells can be sorted using a variety of flow cytometry methodologies well known to those of ordinary skill in the art. Flow cytometry simultaneously measures and then analyzes multiple physical characteristics of single cells. Exemplary properties measured include cell size, relative granularity or internal complexity, and relative fluorescence intensity. The characteristics of each cell are based on its light scattering and fluorescent properties, which is analyzed to provide information about subpopulations within the sample.

In one embodiment, forward-scattered light (FSC) and side-scattered light (SSC) data are collected on the sorted antigen-specific cells. FSC is proportional to cell-surface area or size. As a measurement of mostly diffracted light, FSC provides a suitable method of detecting particles greater than a given size independent of their fluorescence. SSC is proportional to cell granularity or internal complexity, based on a measurement of mostly refracted and reflected light. Correlated measurements of FSC and SSC can allow for differentiation of cell types in a heterogeneous cell population. The staining pattern, e.g., fluorescence, combined with FSC and SSC data, can be used to identify which cells are present in a sample and to count their relative percentages. Then, the cells can be further sorted based on desired properties.

In one embodiment, the stained cells are sorted using flow cytometry, such as fluorescence-activated cell sorting (FACS), magnetic-activated cell sorting (MACS) or microfluidics. The cell sorting may be performed using automated FACS (FACScan™ or BD Influx™, Becton Dickinson or an EPICS Elite™, Beckman Coulter) or MACS technology (autoMACS®) to promote high-throughput and accurate sorting. In one embodiment, the stained antigen-specific cells are single cell sorted into RT-PCR reaction medium, which facilitates amplification of the antigen-specific variable sequences of the antibody expressed by the sorted B cells.

Preferably, in addition to sorting the cells based on labeling, the cells are further sorted using gating, which sets a numerical or graphical boundary to define the characteristics of cells to include for further analysis. For example, a gate can be drawn around the population of interest. A gate or a region is a boundary drawn around a subpopulation to isolate events for analysis or sorting. Based on FSC or cell size, a gate can be set on the FSC vs SSC plot to allow analysis only of cells of a desired size. In one embodiment, B cells sorted using the negative antigen-specific selection or the positive antigen-specific selection are further sorted by FSC/SSC gating. The gated subpopulation of antigen-specific B cells have a larger, less granular phenotype which correlates with improved amplification rates.

Gating parameters can be based on parameters defined by unstained cell populations of similar composition. In particular, gates for positive selection (B-cell staining), negative selection (EL4 feeder cell staining) and viability are constructed based on the auto-fluorescence of the unstained population. Preferably, gates based on unstained populations have little to no percentage of the cell population falling inside the projected gates. Additionally, gates based on physical parameters can be constructed based on a unique population, e.g., identified as larger and less granular than the majority of cells in the population (presumed to be EL4 feeder cells). B-cell culture wells that are not of interest by functional assay of culture supernatant are harvested and processed alongside wells of interest without the addition of cell staining reagents.

Cloning the Identified Antigen-Specific Antibody or Variant Thereof

The present invention also provides a method for cloning antigen-specific antibody sequences, i.e., V_{H} and/or V_{L} regions, contained in the antibody expressed by an antigen-specific B cell that optionally possesses at least one desired functional property such as affinity, avidity, cytolytic activity and the like. In particular, the methods provided herein optionally include a step of producing antibodies from at least one antigen-specific cell from the enriched cell population by amplifying the antigen-specific variable sequences of the antibody expressed by the sorted B cells, sequencing the nucleic acids, and expressing the nucleic acids or a variant thereof encoding the antigen-specific antibody variable sequences to produce an antibody polypeptide. Methods of producing antibodies in vitro are well known in the art, and any suitable method can be employed.

Typically, the inventive methods further comprise additional steps of isolating and sequencing, in whole or in part, the polypeptide and nucleic acid sequences encoding the desired antibody. Antibody coding sequences of interest include those encoded by the nucleic acid and amino acid sequences identified from the isolated antigen-specific cells, as well as nucleic acids that, by virtue of the degeneracy of the genetic code, are not identical in sequence to the identified nucleic acids, and variants thereof.

Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

These identified nucleic acid sequences or modified versions or portions thereof can be expressed in desired host cells in order to produce recombinant antibodies to a desired antigen.

As discussed above, these methods also include cell staining and sorting steps to select antigen-specific cells with an increased rate of amplification, e.g., more of the isolated antigen-specific B cells express an antigen-specific antibody which can be sequences and recombinantly expressed to confirm binding and/or functional properties.

As noted previously, it is believed that the clonal population of B cells predominantly comprises antibody-secreting B cells producing antibody against the desired antigen. It is also believed based on experimental results obtained with several antigens and with different B cell populations that the clonally produced B cells and the isolated antigen-specific B cells derived therefrom produced according to the invention secrete a monoclonal antibody that is typically of relatively high affinity and moreover is capable of efficiently and reproducibly producing a selection of monoclonal antibodies of greater epitopic variability as compared to other methods of deriving monoclonal antibodies from cultured antigen-specific B cells. In an exemplary embodiment the population of immune cells used in such B cell selection methods will be derived from a rabbit. However, other hosts that produce antibodies, including non-human and human hosts, can alternatively be used as a source of immune B cells. It is believed that the use of rabbits as a source of B cells may enhance the diversity of monoclonal antibodies that may be derived by the inventive methods. Also, the antibody sequences derived from rabbits according to the invention typically possess sequences having a high degree of sequence identity to human antibody sequences making them favored for use in humans since they should possess little antigenicity. In the course of humanization, the final humanized antibody contains a much lower foreign/host residue content, usually restricted to a subset of the host CDR residues that differ dramatically due to their nature versus the human target sequence used in the grafting. This enhances the probability of complete activity recovery in the humanized antibody protein.

The identified antigen-specific cell can be used to derive the corresponding nucleic acid sequences encoding the desired monoclonal antibody. (An AluI digest or direct sequencing of the RT-PCR product can confirm that only a single monoclonal antibody type is produced per well.) As mentioned above, these sequences can be mutated, such as by humanization, in order to render them suitable for use in human medicaments.

Preferably, the method further includes a step of sequencing the polypeptide sequence or the corresponding nucleic acid sequence of the selected antibody. The method also preferably includes a step of producing a recombinant antibody using the sequence, a fragment thereof, or a genetically modified version of the selected antibody. Methods for mutating antibody sequences in order to retain desired properties are well known to those skilled in the art and include humanization, chimerization, production of single chain antibodies; these mutation methods can yield recombinant antibodies possessing desired effector function, immunogenicity, stability, removal or addition of glycosylation, and the like. The recombinant antibody can be produced by any suitable recombinant cell, including, but not limited to mammalian cells such as CHO, COS, BHK, human kidney two-hundred and ninety-three, bacterial cells, yeast cells, plant cells, insect cells, and amphibian cells.

In one embodiment, the antibodies are expressed in haploid or polyploidal yeast cells, i.e., haploid or diploid yeast cells, particularly Pichia, and most typically Pichia pastoris. Prior work has help to establish the yeast Pichia pastoris as a cost-effective platform for producing functional antibodies that are potentially suitable for research, diagnostic, and therapeutic use. See co-owned U.S. Pat. Nos. 7,935,340; 7,927,863 and 8,268,582, each of which is incorporated by reference herein in its entirety. Methods are also known in the literature for design of P. pastoris fermentations for expression of recombinant proteins, with optimization having been described with respect to parameters including cell density, broth volume, substrate feed rate, and the length of each phase of the reaction. See Zhang et al., “Rational Design and Optimization of Fed-Batch and Continuous Fermentations” in Cregg, J. M., Ed., 2007, Pichia Protocols (2nd edition), Methods in Molecular Biology, vol. 389, Humana Press, Totowa, N.J., pgs. 43-63. See also, US 20130045888, entitled “MULTI-COPY STRATEGY FOR HIGH-TITER AND HIGH-PURITY PRODUCTION OF MULTI-SUBUNIT PROTEINS SUCH AS ANTIBODIES IN TRANSFORMED MICROBES SUCH AS PICHIA PASTORIS; and US 20120277408, entitled HIGH-PURITY PRODUCTION OF MULTI-SUBUNIT PROTEINS SUCH AS ANTIBODIES IN TRANSFORMED MICROBES SUCH AS PICHIA PASTORIS”.

Exemplary methods that may be used for manipulation of Pichia pastoris (including methods of culturing, transforming, and mating) are disclosed in Published Applications including U.S. 20080003643, U.S. 20070298500, and U.S. 20060270045, and in Higgins, D. R., and Cregg, J. M., Eds. 1998. Pichia Protocols. Methods in Molecular Biology. Humana Press, Totowa, N. J., and Cregg, J. M., Ed., 2007, Pichia Protocols (2nd edition), Methods in Molecular Biology. Humana Press, Totowa, N.J., each of which is incorporated by reference in its entirety.

In a specific embodiment, the method comprises:
a. obtaining B cells from an animal that has been immunized or naturally exposed to an antigen to yield host antibodies;
b. screening the host antibodies for antigen specificity and neutralization;
c. harvesting B cells from the host;
d. enriching the harvested B cells to create an enriched cell population having an increased frequency of antigen-specific cells;
e. culturing one or more sub-populations from the enriched cell population under conditions that favor the survival of a single B cell to produce a clonal population in at least one culture well;
f. determining whether the clonal population produces an antibody specific to the antigen;
g. isolating some or all of the cells from a putative clonal B cell culture and optionally pooling cells from different putative clonal B cell cultures;
h. staining the isolated cells, which optionally are pooled from different putative clonal B cell cultures, with at least one label that facilitates positive or negative cell sorting;
i. sorting the stained B cells and optionally gating the sorted stained B cells before placing the sorted B cells into RT-PCR reaction medium to facilitate amplification of the antigen-specific variable sequences containing in the antibody expressed by the B cell;
j. sequencing the nucleic acid sequence of the antibody produced by the single B cell;
k. expressing the amplified nucleic acids encoding the antigen-specific antibody variable regions to produce antibody polypeptides; and
l. determining which of the expressed antibody polypeptides bind to the antigen of interest.

The determining step (f) can be effected by screening the antigen-specific cell supernatant of enriched antigen-specific cells for antigen-specificity and/or antibody functionality. Similarly, the determining step (1) can be effected by screening the recombinant antibody for antigen-specificity and/or antibody functionality. In one embodiment, the supernatants of enriched antigen-specific B cells and/or recombinant antibody are screened for antigen-specificity using an ELISA assay.

The inventors have demonstrated that the identification and cloning methods provided herein yield an improved quantity and variety of antibodies for various antigens.

For example, after production of anti-PCSK9 antibodies recombinantly expressed from amplified and sequenced nucleic acids encoding the antigen-specific variable regions of an antibody produced from an enriched B cells with PCSK9 antigen specificity, the recombinant antibodies were screened using an ELISA assay to determine PCSK9 binding affinity and an LDL uptake assay to detect antibodies having the ability to modulate the interaction of PCSK9 with LDLR. Sec, e.g., Lagace et al. (2006) Secreted PCSK9 decreases the number of LDL receptors in hepatocytes and in livers of parabiotic mice. J. Clin. Investig. 116(11): 2995-3005. A total of twenty-one different cell sorts, including both single well and pooled cell sorts, were performed on the positive wells identified using the ELISA screen. Multiple antibody sequences were isolated and resulting recombinant antibodies were produced, e.g., Ab1 and Ab2, using the identification and cloning methods provided herein (see Example 8).

By way of another example, after production of anti-CGRP antibodies recombinantly expressed from amplified and sequenced nucleic acids encoding the antigen-specific variable regions of an antibody produced from an enriched B cells with CGRP antigen specificity, the recombinant antibodies were screened using an ELISA assay to determine the antigen binding affinity. Also, the anti-CORP antibodies can be screened using an assay to detect those antibodies having the ability to block cAMP production in SK-N-MC cells. See, e.g., Zeller et al. (2008) CORP function-blocking antibodies inhibit neurogenic vasodilation without affecting heart rate or arterial blood pressure in rate. Br J Pharmacol 155(7):1093-1103. The ELISA screen identified 35 separate positive wells (i.e., containing antibody supernatant identified as having significant antigen recognition and potency). The 35 wells were pooled together, stained for positive and negative B cell selection, and 19 distinct antibody sequences were generated from the pooled B cells isolated using the RT-PCT methods described herein. Additionally, a single well sort was performed on each of 6 individual positive wells. Five of the six wells were determined to produce an anti-CORP specific antibody. Exemplary anti-CGRP antibodies identified using the identification and cloning methods provided herein include Ab 3 and Ab4 (see Example 9).

Additionally, after production of anti-Target 1 antibodies recombinantly expressed from amplified and sequenced nucleic acids encoding the antigen-specific variable regions of an antibody produced from an enriched B cells with Target 1 antigen specificity, the recombinant antibodies were screened using an ELISA assay for Target 1 binding affinity (see Example 10).

Moreover, after production of anti-NGF antibodies recombinantly expressed from amplified and sequenced nucleic acids encoding the antigen-specific variable regions of an antibody produced from an enriched B cells with NGF antigen specificity, the recombinant antibodies were screened using an ELISA assay to determine antigen binding affinity. The antibodies were also screened using an TF1 cell proliferation assay to detect antibodies having the ability to neutralize NGF-induced proliferation in the TF1 human erythroleukemic cell line. See, e.g., Chevalier et al. (1994) Expression and functionality of the trkA proto-oncogene product/NGF receptor in undifferentiated hematopoietic cells. Blood 83: 1479-1485. The ELISA screen identified several positive wells, of which 54 positive wells were sorted. In particular, 34 wells were sorted in an single well sort and 20 wells were sorted in a pooled sort. A total of 8 different sorts were performed, followed by amplification and sequencing. The resulting antibodies were then screened for functional properties, such as p75 reactivity and/or Ms NGF cross-reactivity), and 15 different antibodies were identified, e.g., Ab 7 and Ab8 (see Example 11).

Finally, after production of anti-Target 2 antibodies recombinantly expressed from amplified and sequenced nucleic acids encoding the antigen-specific variable regions of an antibody produced from an enriched B cells with Target 2 antigen specificity, the recombinant antibodies were screened using an ELISA assay to determine antigen binding affinity (see Example 12).

Improved identification and production of antibodies for IL-6 and TNFα was also previously demonstrated in US 2007/0269868. The methods disclosed therein can easily be modified to include the enrichment methods and antigen-specific cell isolation methods, i.e., staining and sorting steps, provided herein.

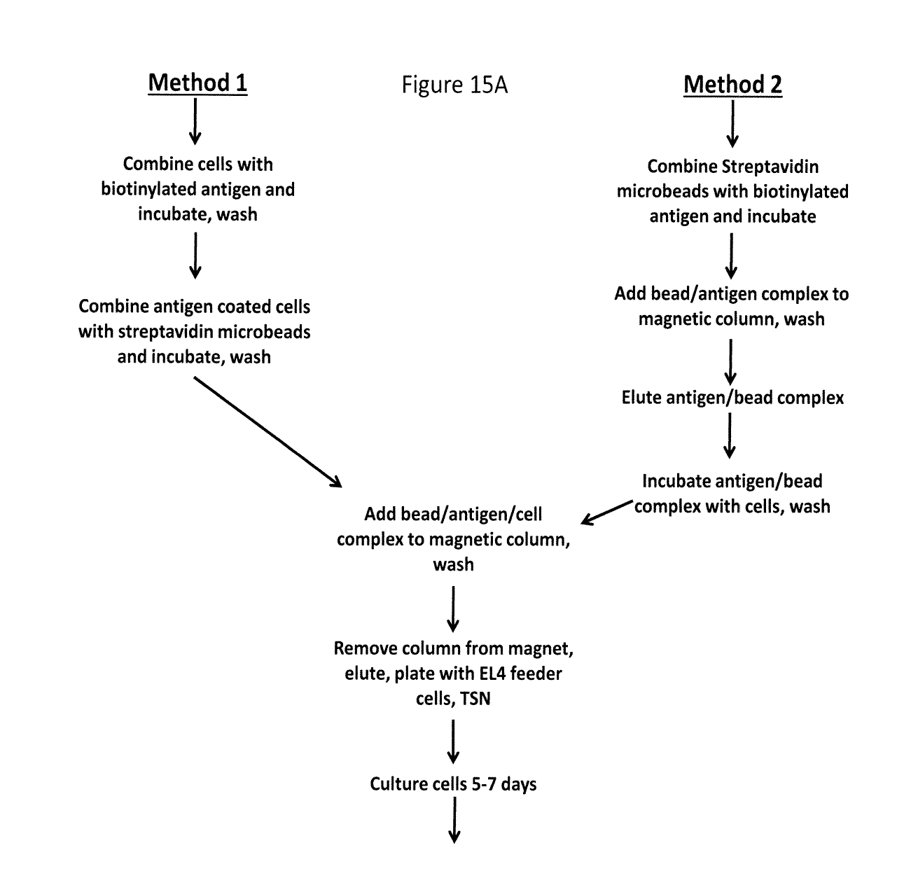

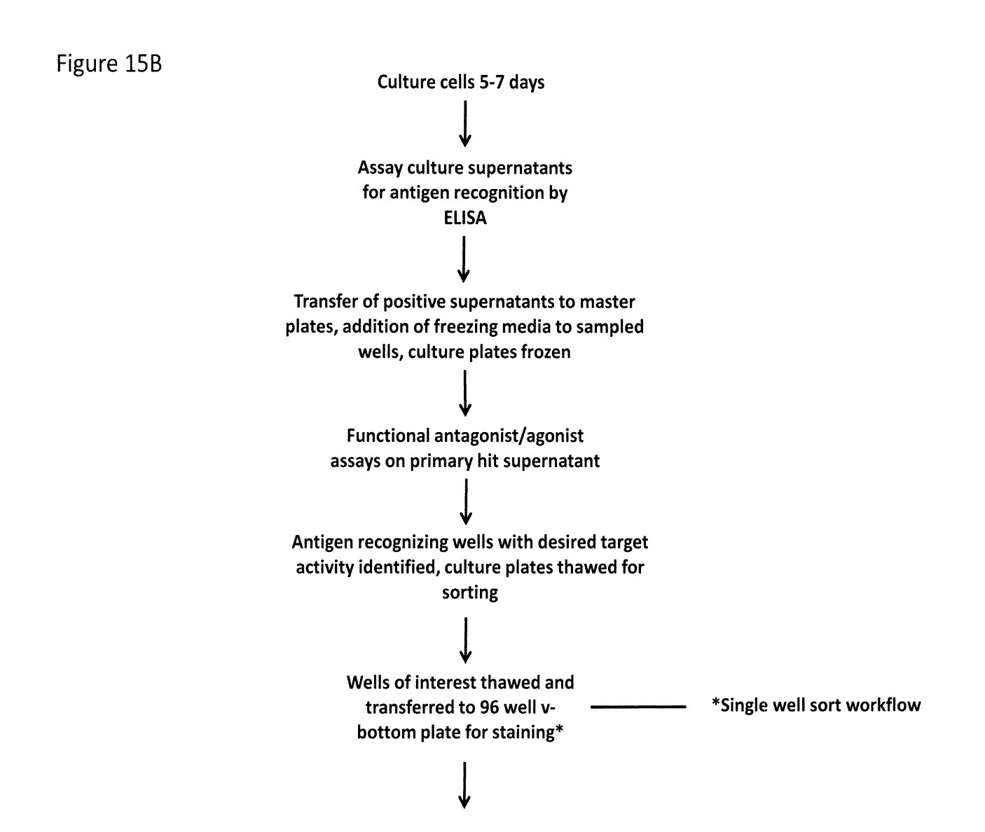

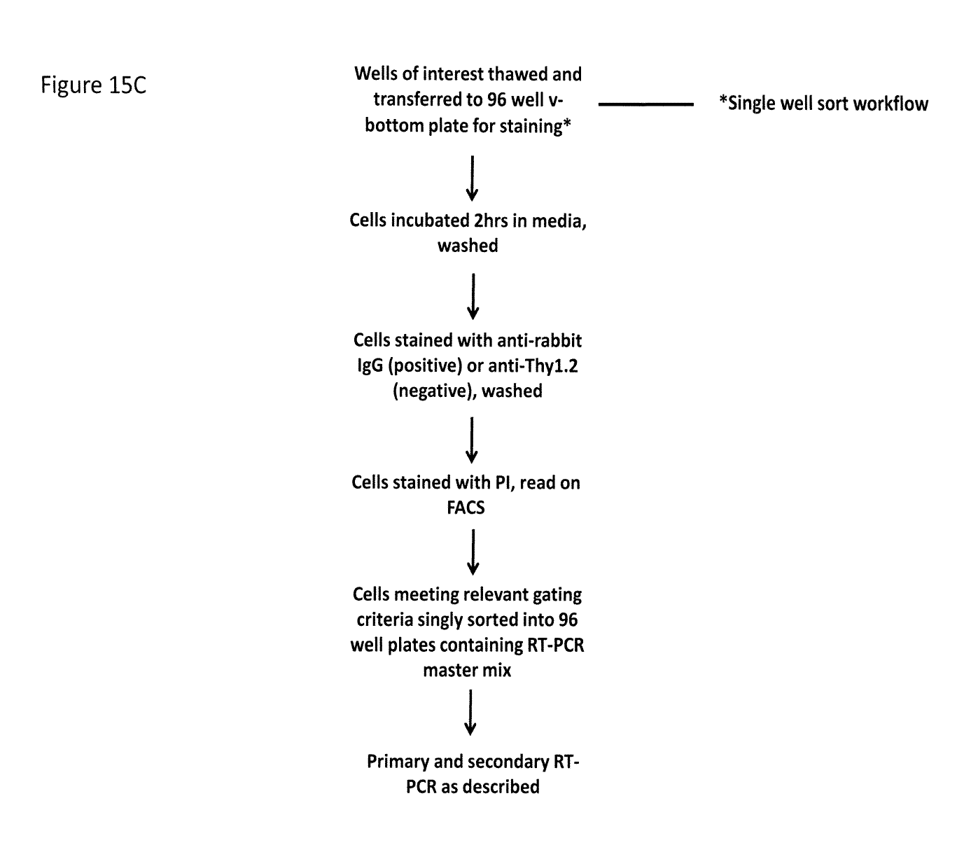

An overview of the inventive B cell selection method is provided in FIGS. 15A-C. These figures illustrate two exemplary embodiments that differ in the initial steps by which the bead/antigen/cell complex is produced. In Method 1 (FIG. 15A, starting from top left corner) collected immune cells (including antigen-specific B cells, e.g., from an animal immunized with or otherwise exposed to the antigen) are initially combined with the biotinylated antigen, and the resultant antigen-coated cells are washed and then contacted with streptavidin microbeads to produce a bead/antigen/cell complex. By contrast, in Method 2 (FIG. 15A, starting from top right corner), streptavidin microbeads are combined with biotinylated antigen, the resultant bead/antigen complex is applied to a magnetic column, washed, and eluted, and then the antigen/bead complexes are incubated with immune cells (including antigen specific B cells, e.g., from an animal immunized with or otherwise exposed to the antigen) to produce bead/antigen/cell. These methods differ in that in Method 1, the antigen initially is not bound to the beads, so that it has greater freedom to bind the B cells; however, some B cells may be lost because retention depends on the biotinylated antigen being captured by a bead. In contrast, in Method 2, by pre-forming the bead-antigen complex, B cells should not be lost due to failure to capture the cell-antigen complex; however, the antigen is constrained by being bound to the bead and so may be less able to bind to the B cell, which can also result in loss of some antigen-specific B cells.

Whether produced by Method 1 or Method 2, the bead/antigen/cell complexes are applied to a magnetic column and washed, after which the column is removed from the magnet to elute the cells (which comprise antigen specific B cells). Cell sorting (such as MACS) can be is utilized to control the number of cells per culture plate, though other techniques could also be utilized. Typically, the cells are plated at varying densities per well in 96 well microtiter plate. Generally, the cells are plated at 10, 25, 50, 100, 250, or 500 cells per well with 10 plates at each density. The range of seeding densities was selected in order to obtain plates whose wells contain clonal populations of antigen-specific B cells after subsequent culturing, i.e., wells containing a single monoclonal antibody specific for the desired antigen. After culturing (with EL4 feeder cells), about 1 to about 100 antigen-specific, clonal IgG-producing B cells are contained in each well.

FIG. 15B shows schematically exemplary culture conditions, functional assays, and antigen recognition assays which may be used to select wells containing cell cultures comprising antigen-specific B cells of interest. These cultures are typically left undisturbed for 5-7 days, during which the B cell populations increase and antibody is secreted into the culture medium; supernatant containing secreted antibody is then collected and evaluated for desired properties, such as antigen binding affinity and/or functional effects. Preferably, supernatants are collected from plates that were seeded at clonal density, i.e., with most wells containing only a single B an antibody specific for the desired antigen. Use of clonal populations increases the efficiency and throughput of screening, because the binding and functional results obtained by testing the supernatant of a given well will likely be the result of a single antibody (as opposed to a combination of antibodies), and moreover that antibody can be more efficiently cloned if the well contains a clonal B cell population, without the need to clone and test multiple independent antibodies.

Supernatants that contain antigen-recognizing antibodies—for example, identified by ELISA—can be transferred to a separate plate for further use, and optionally frozen. The remaining cells can optionally be frozen, e.g., at −80° C., which can be conveniently effected in the same plate in which they were cultured. Further testing can be conducted on supernatants that contained antigen-recognizing antibodies, such as functional assays for a desired activity (e.g., agonist or antagonist activity on the target), and/or further tests of specificity (e.g., specific binding to a desired antigen but not to another antigen such as a similar polypeptide). Wells that produced an antibody having the desired functional properties can be thawed to permit amplification of the antibody-encoding sequences.

FIG. 15C depicts schematically the sorting of antigen-specific B cells to obtain individual cells for amplification and recovery of antibody coding sequences. Thawed cells are stained with anti-rabbit IgG (positive staining, i.e., to stain antibody-producing B cells) or anti-Thy-1.2 (negative staining, i.e., to stain non-antibody cells). The cells are also stained for viability using propidium iodide. Using the positive or negative staining method, PI staining, and physical gating criteria (further described herein), B cells are isolated from EL-4 and other cells contained in the cultures and sorted into individual wells. Antibody-encoding nucleic acids are then amplified by RT-PCR and recovered for cloning, sequencing, or other further use.

To further articulate the invention described above, we provide the following non-limiting examples.

### EXAMPLES

### Example 1

### Production of Enriched Antigen-Specific B Cell Antibody Culture

Panels of antibodies are derived by immunizing traditional antibody host animals to exploit the native immune response to a target antigen of interest. Typically, the host used for immunization is a rabbit or other host that produces antibodies using a similar maturation process and provides for a population of antigen-specific B cells producing antibodies of comparable diversity, e.g., epitopic diversity. The initial antigen immunization can be conducted using complete Freund's adjuvant (CFA), and the subsequent boosts effected with incomplete adjuvant. At about 50-60 days after immunization, preferably at day 55, antibody titers are tested, and the Antibody Selection (ABS) process is initiated if appropriate titers are established. The two key criteria for ABS initiation are potent antigen recognition and function-modifying activity in the polyclonal sera.

At the time positive antibody titers are established, animals are sacrificed and B cell sources isolated. These sources include: the spleen, lymph nodes, bone marrow, and peripheral blood mononuclear cells (PBMCs). Single cell suspensions are generated, and the cell suspensions are washed to make them compatible for low temperature long term storage. The cells are then typically frozen.

To initiate the antibody identification process, a small fraction of the frozen cell suspensions are thawed, washed, and placed in tissue culture media. These suspensions are then mixed with a biotinylated form of the antigen that was used to generate the animal immune response, and antigen-specific cells are recovered using the Miltenyi magnetic bead cell selection methodology. Specific enrichment is conducted using streptavidin beads. For example, the cell preparations are combined with biotinylated antigen and streptavidin beads, passed over a column such that the antigen-specific B cells bind to the column, the bound B cells are then eluted. The enriched population is recovered and progressed in the next phase of specific B cell isolation.

### Example 2

### Production of Clonal, Antigen-Specific B Cell-Containing Culture

Enriched B cells produced according to Example 1 are then plated at varying cell densities per well in a 96 well microtiter plate. Generally, this is at 10, 25, 50, 100, 250, or 500 cells per well with 10 plates per group. Preferably, about 1 to about 100 antigen-specific, clonal IgG-producing B cells are plated per well. The media is supplemented with 1-4% activated rabbit T cell conditioned media along with about 50K frozen irradiated EL4 (EL4B) feeder cells. These cultures are left undisturbed for 5-7 days at which time supernatant containing secreted antibody is collected and evaluated for target properties in a separate assay setting. The remaining supernatant is left intact, and the plate is frozen at −80° C. Under these conditions, the culture process typically results in wells containing a mixed cell population that comprises a clonal population of antigen-specific B cells, i.e., an individual well will only contain a single monoclonal antibody specific to the desired antigen.

### Example 3

### Screening of Antibody Supernatants for Monoclonal Antibody of Desired Specificity and/or Functional Properties

Antibody-containing supernatants derived from the well containing a clonal antigen-specific B cell population produced according to Example 2 are initially screened for antigen recognition using ELISA methods. This includes selective antigen immobilization (e.g., biotinylated antigen capture by streptavidin coated plate), non-specific antigen plate coating, or alternatively, through an antigen build-up strategy (e.g., selective antigen capture followed by binding partner addition to generate a heteromeric protein-antigen complex). For example, the antibody-containing supernatants from B cells obtained from rabbits (either naturally exposed to an antigen or immunized with an antigen) are added to a streptavidin plate coated with biotin-modified antigen or plates coated with unmodified antigen and antigen-specific IgG production is detected with an anti-rabbit IgG. Similarly, the antibody-containing supernatants from B cells obtained from rabbits are added to plates coated with anti-rabbit Fab to detect total IgG production. Detection of antigen-specific IgG production by B cells obtained from another host animal, e.g., mouse, rat, non-human primate or human, is performed using the corresponding anti-species IgG, e.g., anti-mouse IgG, anti-rat IgG, anti-non-human primate IgG or anti-human IgG.

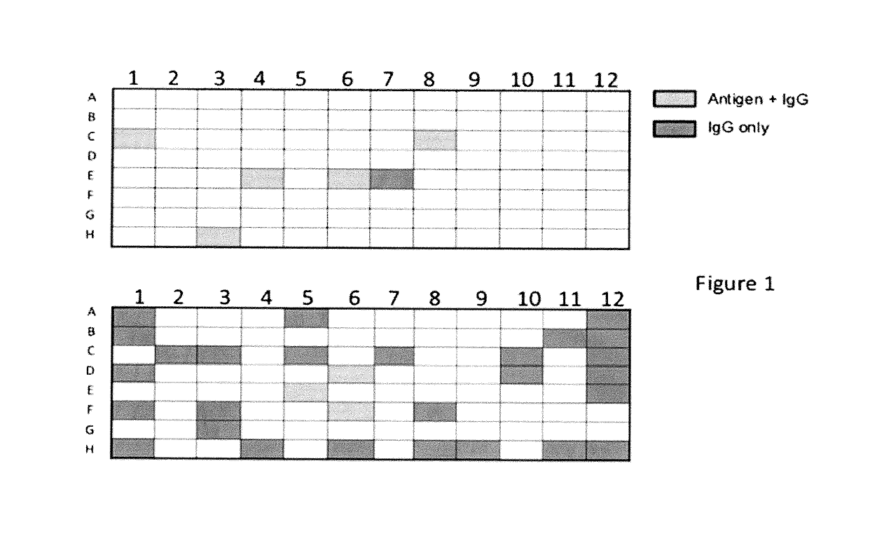

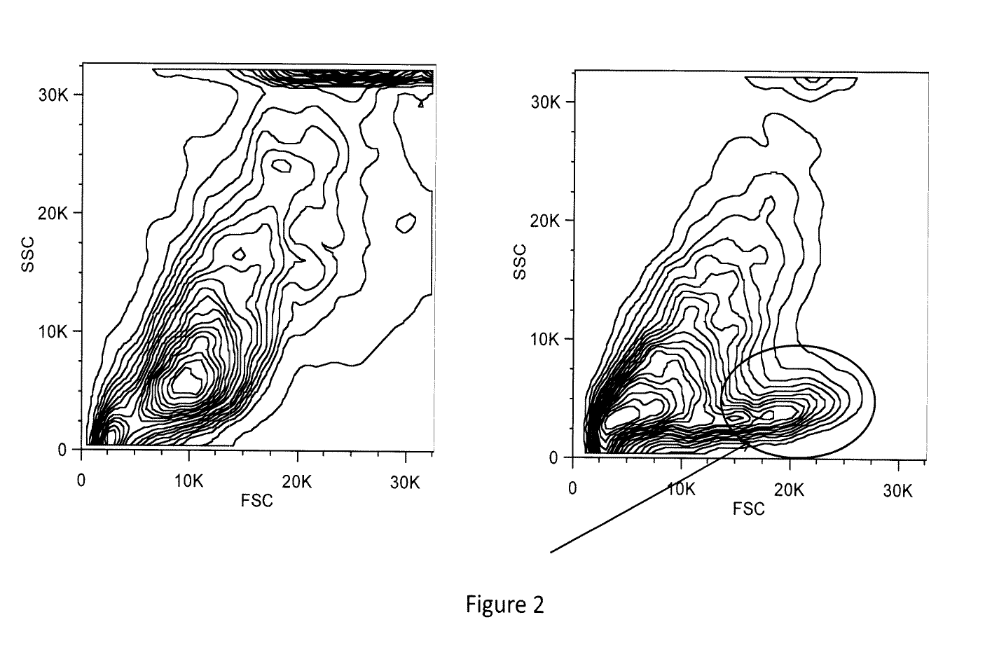

The ratio of antigen-specific wells to total IgG (non-specific) wells is an indicator of enrichment and clonality. In particular, cultures established with well enriched antigen-specific B cells produce predominantly antigen-specific wells (see FIG. 1, top panel), whereas cultures established with poorly enriched antigen-specific B cells show poor correlation between antigen-specific wells and non-specific wells (see FIG. 1, bottom panel).

Antigen-positive well supernatants of enriched B cells are then optionally tested in a function-modifying assay that is strictly dependent on the ligand. One such example is an in vitro protein-protein interaction assay that recreates the natural interaction of the antigen ligand with recombinant receptor protein. Alternatively, a cell-based response that is ligand dependent and easily monitored (e.g., proliferation response) is utilized.

Supernatant that displays significant antigen recognition and potency is deemed a positive well. Cells derived from the original positive well are then transitioned to the antibody recovery phase.

### Example 4

### Isolation of Antigen-Specific B Cells

A single antigen-specific B cell is recovered from a well that contains a clonal population of antigen-specific B cells (produced according to Examples 2 and 3), which secrete a single antibody sequence. Generally, the B cells present in the well are stained with one or more markers for negative selection or positive selection of antigen-specific B cells and the stained cells are sorted directly or indirectly into a RT-PCR master mix for amplification and subsequent sequencing of the antigen-specific variable heavy and/or variable light chain antibody sequences of the antibody expressed by the isolated B cell.

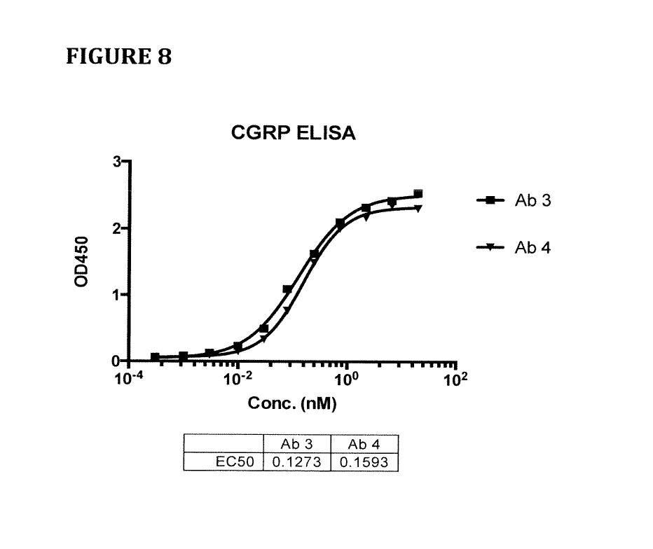

Cell sorter gating was established through the use of control culture wells that are similar in composition to pooled wells or single wells of interest. The gating cell samples were thawed and stained alongside target wells. Initial gates are established on unstained or blank populations. The stained control samples are then run on FACS (BD Influx) and gates are confirmed for EL4 exclusion (CD90.2 positive), B cell inclusion (IgG positive), viability (PI negative) and physical parameters (FSC/SSC) that differentiate B cells from the murine EL4 cells. The latter gate can be established in absence of stain, as it is based on a physical population (size/granularity) that differs from the EL4 cells in culture. Once gates are established, the samples consisting of cells from individual wells or cells pooled from multiple wells are run and EL4 negative/IgG positive, viable cells, preferably of a consistent physical (FSC/SSC) population, are sorted individually into wells of a 96 well plate pre-loaded with RT-PCR master mix. See, FIG. 8. Alternatively, autoMACS® or other MACS® cell sorting technology may be used, e.g., about 50-nm superparamagnetic microbeads, columns and separators for manual or automatic cell sorting and separation. Sorted plates are removed from the sorter and transferred directly to either thermocyclers or −80° C. for RT-PCR amplification of VH and/or VL regions of interest.

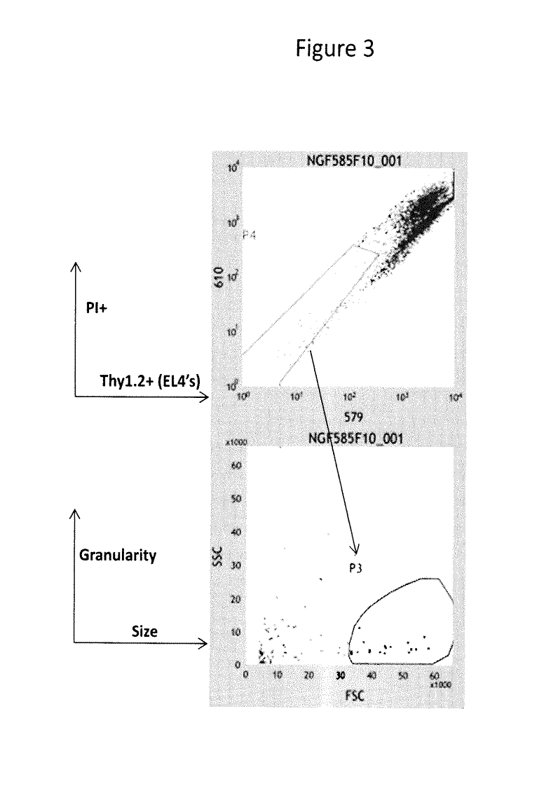

For negative selection of the antigen-specific B cell, cells were stained at 2-10 μg/ml with fluorescent-labeled antibody specific for murine EL4 cells (CD90.2, BD Biosciences, 553014) present in the cell mixture. Cells were stained for approximately 20 minutes at room temperature and following the incubation were washed 2× with up to 2 milliliters of FACS buffer. After washing, cells were re-suspended at approximately 1×10E6 (one million) cells per milliliter FACS buffer. Once re-suspended, Propidium Iodide (BD Biosciences, 556463) was added at 0.2-0.5 μg/ml to identify dead cells in the mixture. Cells that did not stain positive for Thy1.2 and PI were selected (see FIG. 3, top panel). Optionally, a subpopulation of the Thy1.2/PI negative cell population having a larger, less granular phenotype is selected using a final FSC/SSC gate (see FIG. 3, bottom panel).

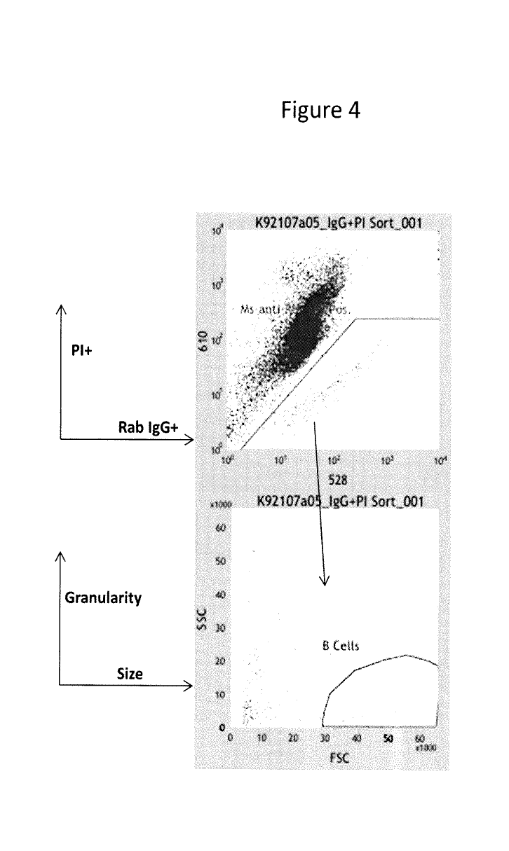

For positive selection of the antigen-specific B cell, cells were stained at 2-10 μg/ml with fluorescent-labeled antibody specific for Rabbit B cells (anti-Rabbit IgG Fc, Creative Diagnostics, DMAB4779) present in the cell mixture. Cells were stained for approximately 20 minutes at room temperature and following the incubation were washed 2× with up to 2 milliliters of FACS buffer. After washing, cells were re-suspended at approximately 1×10E6 (one million) cells per milliliter FACS buffer. Once re-suspended, Propidium Iodide (BD Biosciences, 556463) was added at 0.2-0.5 μg/ml to identify dead cells in the mixture. Cells that did stain positive for Rabbit IgG and negative for PI were selected (see FIG. 4, top panel). Optionally, a subpopulation of the Rabbit IgG+ and PI− cell population having a larger, less granular phenotype than the main population is selected using a final FSC/SSC gate (see FIG. 4, bottom panel).

The stained B cells from an individual well can be sorted (single well sort). Typically, 10 to 20 wells are stained individually and sorted individually prior to amplification. Alternatively, the stained B cells from multiple wells may be pooled together and sorted (pooled cell sort). For example, the contents of at least 100 separate wells are thawed and pooled together for staining as a single sample. The pooled stained cells are then sorted, amplified and sequenced.

For single well sorting, plates containing wells of interest were removed from −80° C., and the cells from each well were recovered using five washes of 200 microliters of medium (10% RPMI complete, 55 μM BME) per well. The recovered cells were pelleted by centrifugation and the supernatant was carefully removed. Cells from each well were then re-suspended in 200 microliters of medium in a FACS tube. Cells were incubated for 120 minutes at 37 degrees C. with the cap loosely secured. Following incubation, cells were pelleted by centrifugation and washed with up to 2 milliliters FACS buffer (Dulbecco's PBS w/2% FBS) and re-suspended in 100 μl of FACS buffer.

For pooled cell sorting, plates containing wells of interest were removed from −80° C., and the cells from each well were recovered using five washes of 200 microliters of medium (10% RPMI complete, 55 μM BME) per well. The recovered cells were pelleted by centrifugation and the supernatant was carefully removed. Cells pooled were then re-suspended in 200 microliters of medium per well (X wells·200 μL=total volume) and transferred to a tissue culture flask of appropriate volume. Cells were incubated for 120 minutes at 37 degrees C. Following incubation, cells were pelleted by centrifugation and washed with up to 2 milliliters FACS buffer (Dulbecco's PBS w/2% FBS) and re-suspended in 100 μl of FACS buffer per well pooled.

### Example 5

### Isolation of Antibody Sequences From Antigen-Specific B Cell

Antibody sequences are recovered using a combined RT-PCR based method from a single isolated B-cell produced according to Example 4. Primers are designed to anneal in conserved and constant regions of the target immunoglobulin genes (heavy and light), such as rabbit immunoglobulin sequences, and a two-step nested PCR recovery step is used to obtain the antibody sequence. A synthetic control RNA sample generated from the expression vector, e.g., T7, is used as a positive control. Amplicons from each well are analyzed for recovery by Pico Green analysis and optionally for size integrity (e.g., by electrophoresis). The PCR product is sequenced directly in a multi-well plate format, e.g., 96 well plate, and stained using Pico green. Alternatively, the resulting fragments are digested with AluI to fingerprint the sequence clonality. Identical sequences display a common fragmentation pattern in their electrophoretic analysis. Significantly, this common fragmentation pattern which proves cell clonality is generally observed even in the wells originally plated up to 1000 cells/well. The resulting AluI digestion product is analyzed using gel electrophoresis and ethidium bromide staining. The original heavy and light chain amplicon fragments are then restriction enzyme digested with HindIII and XhoI or HindIII and BsiwI to prepare the respective pieces of DNA for cloning. The resulting digestions are then ligated into an expression vector and transformed into bacteria for plasmid propagation and production. Colonies are selected for sequence characterization.

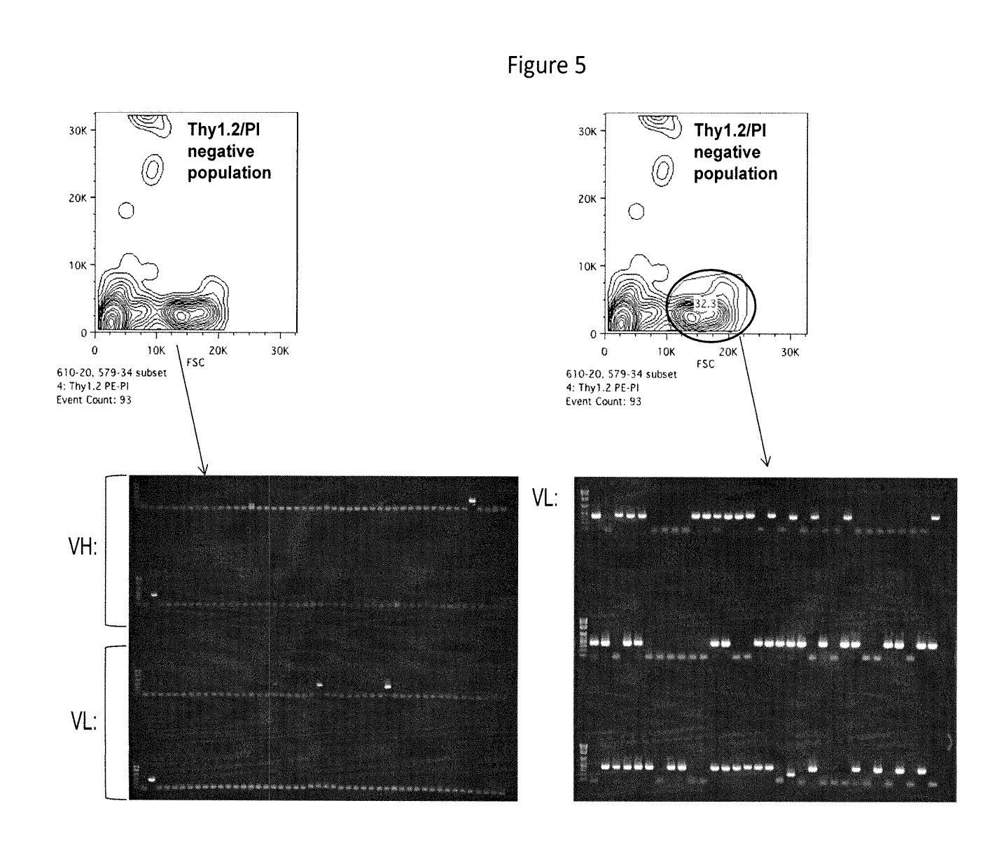

Typically, antigen-expressing specific B cells sorted with the final FSC/SSC gate, such that the cells have a consistent phenotype of larger, less granular cells, have a better than average amplification success. For example, 26 of 88 FSC/SSC gated Thy1.2/PI negative B cells tested displayed the desired fragmentation pattern, compared to 1 of 88 Thy1.2/PI negative B cells (without the final FSC/SSC gate). See, FIG. 5.

Additionally, FSC/SSC gated anti-CGRP antibody producing B cells assayed in a pooled sort that stained for negative and positive selection, demonstrated improved amplification rates. In particular, 32 separate wells from CGRP culture plates containing B cell supernatant that tested positive for antigen-specificity using ELISA were pooled together and subsets of the pooled population were stained by negative selection (Thy1.2) and positive selection (anti-Rabbit Fc monoclonal or anti-Rabbit Fe polyclonal). To begin, 96 wells from the negative selection staining were sorted and 69.8% of the FSC/SSC gated B cells resulted in amplification of the VH and VL chain. Additionally, 80 wells from the anti-Rabbit Fe monoclonal positive selection staining were sorted and 91.3% of the FSC/SSC gated B cells resulted in amplification of the VH and VL chain. Lastly, 96 wells from anti-Rabbit Fc polyclonal staining were sorted and 80.2% and 84.4% of FSC/SSC gated B cells resulted in amplification of the VH and VL chain, respectively.

### Example 6

### Recombinant Production of Monoclonal Antibody of Desired Antigen Specificity and/or Functional Properties

Correct full-length antibody sequences for each well containing a single monoclonal antibody is established and miniprep DNA is prepared using Qiagen solid-phase methodology. This DNA is then used to transfect mammalian cells to produce recombinant full-length antibody. Crude antibody product is tested for antigen recognition and functional properties to confirm the original characteristics are found in the recombinant antibody protein. Where appropriate, large-scale transient mammalian transfections are completed, and antibody is purified through Protein A affinity chromatography. K_{D} is assessed using standard methods (e.g., ProteOn or Biacore®) as well as IC₅₀ in a potency assay.

### Example 7

### Recovery of Isolated B Cell Variable Light and Heavy Chain Sequence and Expression of Recombinant Antibody

The coding sequence for the light and heavy chain were recovered from the single B cells, which had been previously stored at −70° C. A two step reverse transcription polymerase chain reaction (RT-PCR) process was employed. In Step 1, the RNA encoding the areas of interest was recovered by a standard RT-based method that was subsequently amplified. Step 2 was conducted via a nested primer PCR amplification that generates the appropriate DNA fragments for directional cloning into the expression vector: Light chain: HindIII/BsiWI and Heavy chain: HindIII/XhoI. The specific sequences for this recovery process were derived from sequence analysis of the host animal genome. A major source of novel sequence is the rabbit, as well as the mouse and rat. The primer sequences were:

| | Primer | |
|---|---|---|
| | SEQ ID NO. | Sequence (5′ to 3′) |
| Vk sense | 1 | AG[GA]ACCCAGCATGGACA[CT] |
| outer | | [CGA]A |
| Vk sense | 2 | GATATCAAGCTTCGAATCGACATGGAC |
| inner | | ACGAGGGCCCCC (HindIII/SfuI) |
| Ck anti- | 3 | GGA[TC][AG]G[AT]ATTTATT[CT] |
| sense | | GCCAC[GA]CACA |
| outer | | |
| Ck anti- | 4 | TCTAGACGTACGTTTGACCACCACCTC |
| sense | | GGTCCCTC (BsiWI) |
| inner 1 | | |
| Ck anti- | 5 | TCTAGACGTACGTAGGATCTCCAGCTC |
| sense | | GGTCCC (BsiWI) |
| inner 2 | | |
| Ck anti- | 6 | TCTAGACGTACGTTTGATTTCCACATT |
| sense | | GGTGCCAGC (BsiWI) |
| inner 3 | | |
| VH sense | 7 | AGAC[AG]CTCACCATGGAGACT |
| outer | | |
| VH sense | 8 | GATATCAAGCTTACGCTCACCATGGAG |
| inner | | ACTGGGC (HindIII) |
| Cg CH1 | 9 | ACTGGCTCCGGGAGGTA |
| anti-sense | | |
| outer | | |
| Cg CH1 | 10 | CGCGCGCTCGAGACGGTGAC[CG]AGG |
| anti-sense | | GT[CG]CC[CT][GT]GGCCCC |
| inner | | (XhoI) |

Cloned cDNAs were then ligated into two distinct mammalian expression vectors (kappa light chain constant and gamma-1 (γ-1) heavy chain constant) that enable expression of the recombinant light and heavy chain. These constructs were made in frame and incorporated the natural signal sequence included in the sequence recovery. Large scale DNA preparations were made for each expression plasmid, and transient production of full length rabbit/human chimeric antibody was conducted by transfection using both plasmids into human kidney two-hundred and ninety-three cells. After 5 days in culture, the resulting cells were removed by centrifugation, and the condition medium was tested directly for antigen recognition, or the recombinant antibody was affinity purified via Protein A chromatography.

The antibody was then tested for antigen recognition using the ELISA method described above. In addition, for the purified antibody, the K_{D} was established by a ForteBio Octet or BioRad or ProteOn measurement. Finally, the original function-modifying properties attributed to the particular well associated with the recovered sequence were tested.

### Experimental Method for Light and Heavy Chain Sequence Recovery.

The method is based on the technology described in the manufacturer's description for the Qiagen One Step RT-PCR kit. A common master mix was prepared and included RNasin (Promega) to prevent RNA degradation. 50 μL of RT-PCR master mix containing 0.58 μM of each step 1 primer (Primer SEQ ID NOs.: 1, 3, 7, and 9) was added to the 250 μL eppendorf tube containing previously recovered frozen cell and carefully mixed on ice. The One Step RT-PCR was performed with the following cycle scheme: (1) 50° C., 30 minutes; (2) 95° C., 15 minutes; (3) 94° C., 30 seconds; (4) 54° C., 30 seconds; (5) 72° C., 1 minute; (6) go to step 3, 35 cycles total; (7) 72° C., 3 minutes; and (8) 4° C., hold.

When these cycles were completed, the secondary PCR amplification was conducted in separate reactions to recover the light and heavy chain variable regions using 1.5 μL of the primary RT-PCR reaction. A KOD polymerase driven amplification (Novagen) with 0.4 μM of secondary nested PCR primers light chain (Primer SEQ ID NOs.: 2 and 4; SEQ ID NOs.:2 and 5; or SEQ ID NOs.:2 and 6) and heavy chain (Primer SEQ ID NOs.: 8 and 10) using the following cycle scheme: (1) 94° C., 2 minutes; (2) 94° C., 30 seconds; (3) 60° C., 30 seconds; (4) 72° C., 45 seconds; (5) go to step 2, 35 cycles total; (6) 72° C., 3 minutes; and (7) 4° C., hold.

Upon completion of the secondary amplification, 10 μL of the reaction was removed and analyzed by 2% TAE agarose gel electrophoresis. The remaining 40 μL of the reaction were purified via Qiagcn Qiaquick™ PCR Clean-up kit and eluted in 75 μL.

These amplicons were subsequently digested with HindIII/BsiWI in the case of light chain and HindIII/XhoI for the heavy chain using the following conditions: 10 μL Purified PCR product, 3 μL 10× New England Biolabs restriction enzyme buffer 2, 0.5 μL HindIII (5U), and 0.5 μL BsiWI (5U) or 0.5 μL XhoI for 60 minutes at 37° C. followed by 30 minutes at 55° C. The digests were purified via Qiagen Qiaquick™ PCR method. These were subsequently ligated into the appropriate expression vector. 2 μL of this reaction was then used to transform either TOP10 (Invitrogen) or XL-10 (Stratagene), and the transformed cells were plated on LB/Kanamycin (50 μg/mL).

The resulting colonies were screened for inserts via a PCR screening method employing the following primers:

| | Primer | |
|---|---|---|
| | SEQ ID | |
| | NO. | Sequence (5′ to 3′) |
| Vector | 11 | GCGCGCCACCAGACATAATAGCT |
| Heavy Chain | 12 | AGCCCAAGGTCACCGTGCTAGAG |
| Light Chain | 13 | GTATTTATTCGCCACACACACACGATG |

Colonies were picked into 60 μL LB/kanamycin and incubated for up to 30 minutes. At 30 min, approximately 1 μL was removed and used in a standard 30 μL KOD amplification reaction (Novagen) containing 2 μM of the primer pair SEQ ID NOs.: 11 and 12 for the heavy chain and SEQ ID NOs.: 11 and 13 for the light chain. The amplification scheme was as follows: (1) 96° C., 2 minutes; (2) 96° C., 20 seconds; (3) 68° C., 25 seconds; (4) go to 2, repeat for 40 cycles total; and (5) 68° C., 2 minutes.

Amplification was verified using Pico Green analysis. The sample was then sequenced directly in a multi-well plate format, e.g., 96 well plate.

### Example 8

### Preparation of Antibodies that Bind Human PCSK9

By using the antibody selection protocol described herein, an extensive panel of antibodies can be generated, including Ab1 and Ab2 which are distinct antibodies with specificity for human PCSK9 (huPCSK9). The antibodies have high affinity towards huPCSK9 (e.g., about 10 to about 900 pM K_{D}) and demonstrate potent antagonism of huPCSK9 in cell-based screening systems (HepG2). Furthermore, the collection of antibodies displayed distinct modes of antagonism toward huPCSK9-driven processes.

Immunization Strategy:

Rabbits were immunized with huPCSK9 (R&D). Immunization consisted of a first subcutaneous (sc) injection of 100 μg in complete Freund's adjuvant (CFA) (Sigma) followed by two boosts, two weeks apart, of 50 μg each in incomplete Freund's adjuvant (IFA) (Sigma). Animals were bled on day 55, and serum titers were determined by ELISA (antigen recognition).

Antibody Selection Titer Assessment:

To identify and characterize antibodies that bind to human huPCSK9, antibody-containing solutions were tested by ELISA. Briefly, neutravidin coated plates (Thermo Scientific), were coated with biotinylated human PCSK9 (50 μL per well, 1 μg/mL) diluted in PBS for approximately 1 hour at room temperature or alternatively overnight at 4° C. The plates were then blocked with ELISA buffer for one hour at room temperature and washed using wash buffer (PBS, 0.05% Tween 20). Serum samples tested were serially diluted using ELISA buffer (0.5% fish skin gelatin in PBS pH 7.4). Fifty microliters of diluted serum samples were transferred onto the wells and incubated for one hour at room temperature. After this incubation, the plate was washed with wash buffer. For development, an anti-rabbit specific Fc-HRP (1:5000 dilution in ELISA buffer) was added onto the wells and incubated for 45 minutes at room temperature. After a 3× wash step with wash solution, the plate was developed using TMB substrate for two minutes at room temperature and the reaction was quenched using 0.5M HCl. The well absorbance was read at 450 nm.

Tissue Harvesting:

Once acceptable titers were established, the rabbit(s) were sacrificed. Spleen, lymph nodes, and whole blood were harvested and processed as follows:

Spleen and lymph nodes were processed into a single cell suspension by disassociating the tissue and pushing through sterile wire mesh at 70 μm (Fisher) with a plunger of a 20 cc syringe. Cells were collected in modified RPMI medium described above with low glucose. Cells were washed twice by centrifugation. After the last wash, cell density was determined by staining cells with Trypan Blue and counting using a hempcytometer. Cells were centrifuged at 1500 rpm for 10 minutes; the supernatant was discarded. Cells were resuspended in the appropriate volume of 10% dimethyl sulfoxide (DMSO, Sigma) in FBS (Hyclone) and dispensed at 1 ml/vial. Vials were then stored at −70° C. for 24 hours prior to being placed in a liquid nitrogen (LN₂) tank for long-term storage.

Peripheral blood mononuclear cells (PBMCs) were isolated by centrifuging whole blood for 30 minutes at 2000 rpm, removing plasma, resuspending remaining blood volume to 50 mL with PBS, and splitting volume equally into 2 new 50-mL conical tubes (Corning). 8 mL of Lympholyte Rabbit (Cedarlane) was carefully underlayered below blood mixture and centrifuged 30 minutes at 2000 rpm at room temperature without brakes. After centrifugation, the PBMC layers were carefully removed using a glass Pasteur pipette (VWR), combined, and placed into a clean 50 ml vial. Cells were washed once with PBS by centrifugation at 2000 rpm for 10 minutes at room temperature, and cell density was determined by Trypan Blue staining. After the wash, cells were resuspended in appropriate volume of 10% DMSO/FBS medium and frozen as described above.

B Cell Culture:

On the day of setting up B cell culture, PBMC, splenocyte, or lymph node vials were thawed for use. Vials were removed from LN₂ tank and placed in a 37° C. water bath until thawed. Contents of vials were transferred into 15 mL conical centrifuge tube (Corning) and 10 mL of modified RPMI described above was slowly added to the tube. Cells were centrifuged for 5 minutes at 1-2K rpm, and the supernatant was discarded. Cells were re-suspended in 10 mL of fresh media. Cell density and viability was determined by Trypan Blue staining. Cells were washed again and resuspended in 100 μl Phosphate Buffered Formula [(PBF): Ca/Mg free PBS (Hyclone), 2 mM ethylene-diamine tetraacetic acid (EDTA), 0.5% bovine serum albumin (BSA) (Sigma-biotin free)] per 1E7 cells. During washes the biotinylated antigen was diluted to approximately 5 μg/ml in PBF. Biotinylated antigen was combined with 10-20 μl MACS® streptavidin beads (Milteni) and incubated at 4° C. for 15 minutes. Following incubation, coated beads were passed over pre-wetted MACS® MS column (Milteni) column. The coated beads were rinsed 3 times with 500 μl PBF and eluted in the original volume. Coated beads were combined with thawed cells, mixed, and incubated at 4° C. for 30 minutes. Following incubation, the mixture of cells and beads was passed over the MS column. The column was washed 5 times with 500 μl PBF, removed from magnet and cells were eluted in 0.5-1 mL PBF. The cells were counted and re-suspended in appropriately the volume of modified RPMI described above. Positive selection (enrichment) yielded an average of 1% from the starting cell concentration. A pilot cell screen was established to provide information on cell seeding levels for the culture. Three to four groups of 3 to 10 96-well plates (a total of up to 40 plates) were set at 10, 20, 50 and 100 enriched B cells per seeding density. In addition, each well contained 50K cell/well of irradiated EL-4.B5 cells (5,000 Rads) and an appropriate level of T cell supernatant (ranging from 1-5% depending on preparation) in high glucose modified RPMI medium at a final volume of 250 μl/well. Cultures were incubated for 5 to 7 days at 37° C. in 4% CO₂.

Identification of Selective Antibody Secreting B Cells:

Cultures were tested for antigen recognition and functional activity between days 5 and 7.

B Cell Culture Antigen Recognition Screening:

The same ELISA format described for titer assessment was used for antigen recognition screening except 50 μl of supernatant from the B cell cultures (BCC) wells (all 40 plates) was used as the source of the antibody. The conditioned medium was transferred to antigen-coated plates. After positive wells were identitied by ELISA, the supernatant from the positive B cell culture wells was removed and transferred to a 96-well master plate(s). The original culture plates were then frozen by removing all the supernatant except 40 μl/well and adding 60 μl/well of 16% DMSO in FBS. Plates were wrapped in paper towels to slow freezing and placed at −70° C.

Functional Activity Screening:

Master plates were then screened for functional activity in the huPCSK9-LDLr binding ELISA. Neutravidin plates were coated with biotinylated polyclonal anti-huPCSK9 (R&D) and washed. Following coating, unpurified D374Y huPCSK9 from transiently transfected human kidney two-hundred and ninety-three cells was incubated with B-cell supernatants prior to being added to the wells and allowed to bind. Following an additional wash, recombinant, his-tagged LDLr (R&D) was added for I hour at room temperature. After another wash, an anti-his tag, HRP conjugated antibody (Invitrogen) was added (lot dependent concentration) to detect LDLr binding. After 3 additional washes, 50 ul of TMB was added to develop for 15 minutes followed by 50 μl of 0.5M HCl. Plates were read at 450 nm.

B Cell Recovery-Single Well Sort:

Plates containing wells of interest were removed from −70° C., and the cells from each well were recovered using five washes of 200 microliters of medium (10% RPMI complete, 55 μM BME) per well. The recovered cells were pelleted by centrifugation and the supernatant was carefully removed. Cells from each well were then re-suspended in 200 microliters of medium in a FACS tube. Cells were incubated for 120 minutes at 37 degrees C. (4% CO2) with the cap loosely secured. Following incubation, cells were pelleted by centrifugation and washed with up to 2 milliliters FACS buffer (Dulbecco's PBS w/2% FBS) and re-suspended in 100 ul of FACS buffer.

B Cell Recovery-Pooled Sort:

Plates containing wells of interest were removed from −70° C., and the cells from each well were recovered using five washes of 200 microliters of medium (10% RPMI complete, 55 μM BME) per well. The recovered cells were pooled and pelleted by centrifugation and the supernatant was carefully removed. Cells were then re-suspended in 200 microliters of medium per well, pooled, and transferred to a tissue culture flask of appropriate volume. Cells were incubated for 120 minutes at 37 degrees C. Following incubation, cells were pelleted by centrifugation and washed with up to 2 milliliters FACS buffer (Dulbecco's PBS w/2% FBS) and re-suspended in 100 μl of FACS buffer per well pooled.

B Cell Recovery-Positive Stain:

Cells were stained at 2-10 μg/mL with fluorescent-labeled antibody specific for murine EL4 cells (CD90.2, BD Biosciences, 553014) present in the cell mixture for approximately 20 minutes at room temperature. Following the incubation cells were washed 2× with up to 2 milliliters of FACS buffer. After washing, cells were re-suspended at approximately 1×10E6 (one million) cells per milliliter FACS buffer. Once re-suspended, Propidium Iodide (BD Biosciences, 556463) was added at 0.2-0.5 μg/ml to identify dead cells in the mixture.

B Cell Recovery-Negative Stain:

Cells were stained at 2-10 μg/ml with fluorescent-labeled antibody specific for Rabbit B cells (anti-Rabbit IgG Fe, Creative Diagnostics, DMAB4779) present in the cell mixture. Cells were stained for approximately 20 minutes at room temperature and following the incubation were washed 2× with up to 2 milliliters of FACS buffer. After washing, cells were re-suspended at approximately 1×10E6 (one million) cells per milliliter FACS buffer. Once re-suspended, Propidium Iodide (BD Biosciences, 556463) was added at 0.2-0.5 μg/ml to identify dead cells in the mixture.

B Cell Sorting Method:

Cell sorter gating was established through the use of control culture wells that were similar in composition to pooled wells or single wells of interest. The gating cell samples were thawed and stained along side target wells. Initial gates were established on unstained or blank populations. The stained control samples were then run on FACS (BD Influx) and gates were confirmed for EL4 exclusion (CD90.2 positive/CD90.2+), B cell inclusion (IgG positive/IgG), viability (PI negative/PI−) and physical parameters (FSC/SSC) that differentiate B cells from the murine EL4 cells. The latter gate can be established in absence of stain, as it is based on physical properties (size/granularity) that differentiates the EL4 cells in culture. Once gates were established, the samples consisting of cells from individual wells or cells pooled from multiple wells were run and EL4 negative/IgG positive, viable cells that were of a consistent physical (FSC/SSC) property were sorted individually into wells of a 96 well plate pre-loaded with RT-PCR master mix. Sorted plates were removed from the sorter and transferred directly to either thermocyclers or −80° C. for PCR amplification of V_{H} and V_{L} regions of interest.

Amplification and Sequence Determination of Antibody Sequences from Antigen-Specific B Cells:

Antibody sequences were recovered using a combined RT-PCR based method from a single isolated B-cell. Primers containing restriction enzymes were designed to anneal in conserved and constant regions of the target immunoglobulin genes (heavy and light), such as rabbit immunoglobulin sequences, and a two-step nested PCR recovery was used to amplify the antibody sequence. Amplicons from each well were analyzed for recovery by Pico Green analysis and optionally for size integrity (e.g., by electrophoresis). The resulting fragments were sent for sequence confirmation. Identical antibodies can easily be identified through their sequencing returns. The original heavy and light chain amplicon fragments were then digested using the restriction enzyme sites contained within the PCR primers and cloned into an expression vector. Vector containing subcloned DNA fragments were amplified and purified. Sequence of the subcloned heavy and light chains were verified prior to expression.

Recombinant Production of Monoclonal Antibody of Desired Antigen Specificity and/or Functional Properties:

To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

Antigen-Recognition of Recombinant Antibodies by ELISA:

To characterize recombinant expressed antibodies for their ability to bind to human PCSK9, antibody-containing solutions were tested by ELISA. All incubations were done at room temperature. Briefly, Neutravidin plates (Thermo Scientific) were blocked for lhour with ELISA buffer (PBS, 0.5% fish skin gelatin, 0.05% Tween-20). After blocking, plates were coated with a biotinylated-huPCSK9 containing solution (1 μg/mL in ELISA buffer) for 1 hour. Human PCSK9-coated plates were then washed three times in wash buffer (PBS, 0.05% Tween-20). After coating, the plates were blocked again with ELISA buffer for 1 hour. The blocking solution was removed and the plates were then incubated with a dilution series of the antibody being tested for approximately 1 hour. At the end of this incubation, the plate was washed three times with wash buffer and further incubated with a secondary antibody containing solution (Peroxidase conjugated affinipure F(ab′)2 fragment goat anti-human IgG, Fe fragment specific [Jackson lmmunoresearch]) for approximately 45 minutes and washed three times. Next, a substrate solution (TMB peroxidase substrate, BioFx) was added and incubated for 3 to 5 minutes in the dark. The reaction was stopped by addition of 0.5M HCl and the plate was read at 450 nm in a plate-reader.

Functional Characterization of Recombinant Antibodies by Modulation of LDL-C Uptake by HepG2 Cells:

The ability of anti-PCSK9 antibodies to neutralize the inhibition of LDL-C uptake in HepG2 cells by huPCSK9 was tested in a cell-based assay. HepG2 cells were seeded (30,000 cells/well) in a collagen coated 96 well plate. Twenty-four hours later, the media was replaced with fresh media (MEM) containing 0.5% low lipid FBS. Various concentrations of anti-PCSK9 antibodies were incubated with 3 μg/mL huPCSK9 for 1 hour at room temperature and then added to the HepG2 cells and incubated for 5 hours at 37° C. BODIPY-LDL was added to each well and incubated overnight at 37° C. The media was removed and the cells lysed with RIPA buffer and the amount of BODIPY-LDL taken up by the cells measured on a plate reader (excitation, 485 nm; emission 535 nm).

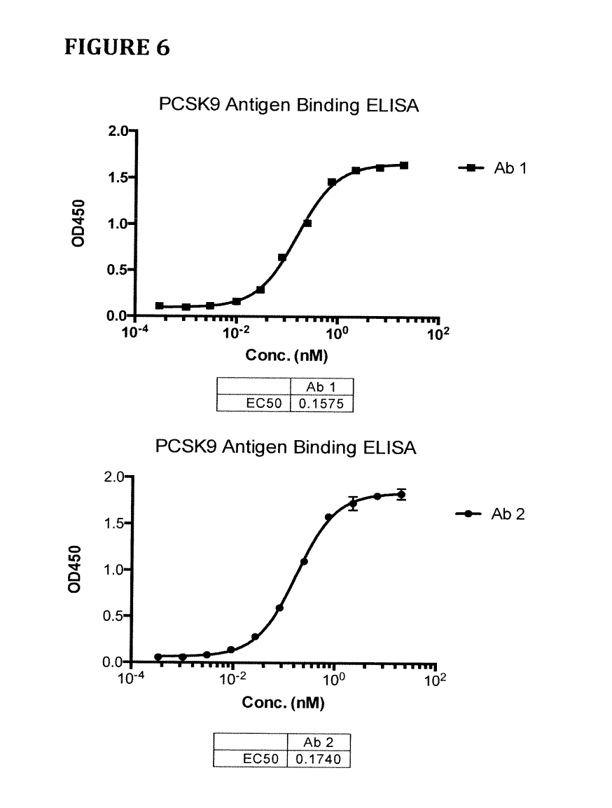

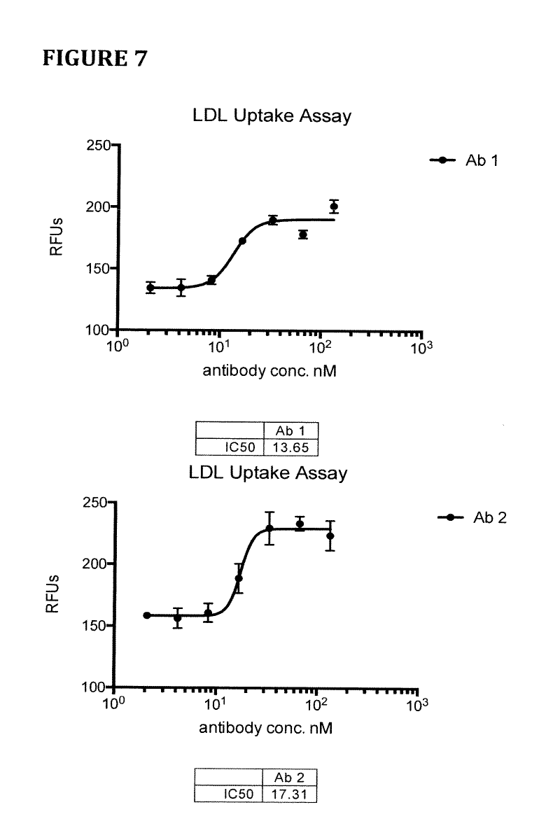

This example demonstrates that multiple anti-PCSK9 antibody sequences were cloned from identified antigen-specific B cells. Exemplary antibodies Ab1 and Ab2 were shown to have high binding affinity for huPCSK9 (see FIG. 6) and demonstrated the ability to block the interaction of huPCSK9 with LDLR (see FIG. 7).

### Example 9

### Preparation of Antibodies that Bind HuCGRP Alpha

By using the antibody selection protocol described herein, one can generate a collection of antibodies that exhibit potent functional antagonism of CGRPα. Antibodies that can selectively bind CGRPα at the N or C terminus of the peptide were identified, including Ab3 and Ab4 which are distinct antibodies with specificity for CGRPα, with the latter comprising the majority of the functional group.

Immunization Strategy:

Rabbits were immunized with human CGRPcr (American Peptides, Sunnyvale Calif. and Bachem, Torrance Calif.). Immunization consisted of a first subcutaneous (sc) injection of 100 μg of antigen mixed with 100 μg of KLH in complete Freund's adjuvant (CFA) (Sigma) followed by two boosts, two weeks apart each containing 50 μg antigen mixed with 50 μg in incomplete Freund's adjuvant (IFA) (Sigma). Animals were bled on day 55, and serum titers were determined by ELISA (antigen recognition) and by inhibition of CGRP driven cAMP increase in SK-N-MC.

ABS Titer Assessment:

Antigen recognition assay was determined for CGRPα by the protocol described for huPCSK9 (see Example 8), with the following exception: neutravidin plates were coated with both N or C-terminally biotinylated CGRP-α at the concentration described above.

Functional Titer Assessment:

To identify and characterize antibodies with functional activity, an inhibition of CGRP driven increase of cAMP levels assay was done using electrochemiluminescence (Meso Scale Discovery, MSD). Briefly, antibody preparations to be tested were serially diluted in MSD assay buffer (Hepes, MgCl2, pH 7.3, 1 mg/mL blocker A, Meso Scale Discovery) in a 96 well round bottom polystyrene plate (Costar). To this plate, human CGRPα was added (10 ng/mL final concentration) diluted in MSD assay buffer and incubated for one hour at 37 C. Appropriate controls were used as suggested by the assay-kit manufacturer. Human neuroepithelioma cells (SK-N-MC, ATCC) were detached using an EDTA solution (5 mM in PBS) and washed using growth media (MEM, 10% FBS, antibiotics) by centrifugation. The cell number was adjusted to 2 million cells per mL in assay buffer, and IBMX (3-Isobutyl-1 Methylxanthine, Sigma) was added to a final concentration of 0.2 mM right before loading cells onto cAMP assay plate. Separately antibody/huCGRPα were mixed and incubated at room temperature for 1 hour. This was then transferred to a MSD cAMP assay plate along with 10 uL of cell suspension described above. This plate was incubated at room temperature with shaking for 30 minutes. Concurrently, the stop solution was prepared (1:200 dilution of TAG label cAMP (MSD) in lysis buffer). 20 ul/well of stop solutions was added to the MSD assay plate, shaken at 20 additional minutes at room temperature. 100 μL of read buffer (MSD; 1:4 dilution in water) was added to each well. The plate was then read using a Sector Imager 2400 (MSD) and the Prism software was used for data fit and IC50 determination.

Tissue Harvesting:

Rabbit spleen, lymph nodes, and whole blood were harvested, processed, and frozen as described above for huPCSK9 (sec Example 8).

B Cell Culture (BCC):

B cell cultures were prepared as described in Example 8 for huPCSK9, except cell enrichment was done using N and C terminally biotinylated huCGRPα.

B Cell Culture Antigen Recognition Screening:

Antigen recognition screening was performed as described above as single points.

Functional Activity Screening:

To determine functional activity contained in B-cell supernatants, a similar procedure to that described for the determination of functional titer of serum samples was used with the following modifications. Briefly, B-cell supernatant (20 μL) were used in place of the diluted polyclonal serum samples.

B Cell Recovery:

The FACS method was performed as described above for huPCSK9 (see Example 8).

Amplification and Sequence Determination of Antibody Sequences from Antigen-Specific B Cells:

Antibody sequences were recovered using the method described above for huPCSK9 (see Example 8).

Recombinant Production of Monoclonal Antibody of Desired Antigen Specificity and/or Functional Properties:

To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

Antigen-Recognition of Recombinant Antibodies by ELISA:

Recombinant antibodies were evaluated for binding as described in titer assessment section. N and C terminally biotinylated ELISAs were run separately to determine binding specificity. The binding affinity for CGRP, as measured by ELISA, was determined for exemplary antibodies Ab3 and Ab4. See FIG. 8.

Functional Characterization of Recombinant Antibodies by Modulation of CGRP Driven Intracellular cAMP Levels:

To characterize recombinant expressed antibody for their ability to inhibit CGRPα mediated increased cellular levels of cAMP assay, an electrochemiluminescence assay-kit (Meso Scale Discovery, MSD) was used. Briefly, antibody preparations to be tested were serially diluted in MSD assay buffer (Hepes, MgCl2, pH 7.3, lmg/mL blocker A, Meso Scale Discovery) in a 96 well round bottom polystyrene plate (Costar). To this plate, human CGRPα was added (25 ng/mL final concentration) diluted in MSD assay buffer and incubated for one hour at 37° C. Appropriate controls were used as suggested by the assay-kit manufacturer. Human neuroepithelioma cells (SK-N-MC, ATCC) were detached using an EDTA solution (5 mM) and washed using growth media (MEM, 10% FBS, antibiotics) by centrifugation. The cell number was adjusted to 2 million cells per mL in assay buffer, and IBMX (3-Isobutyl-1 Methylxanthine, 50 mM Sigma) was added to a final concentration of 0.2 mM right before loading cells onto cAMP assay plate. Separately antibody/huCGRPα were mixed and incubated at room temperature for 1 hour. This was then transferred to a MSD cAMP assay plate along with 10 uL of cell suspension described above. This plate was incubated at room temperature with shaking for 30 minutes. Concurrently, the stop solution was prepared (1:200 dilution of TAG label cAMP (MSD) in lysis buffer). 20 μL/well of stop solutions was added to the MSD assay plate, shaken at 20 additional minutes at room temperature. 100 μL of read buffer (MSD; 1:4 dilution in water) was added to each well. The plate was then read using a Sector Imager 2400 (MSD) and Prism software was used for data fit and IC50 determination.

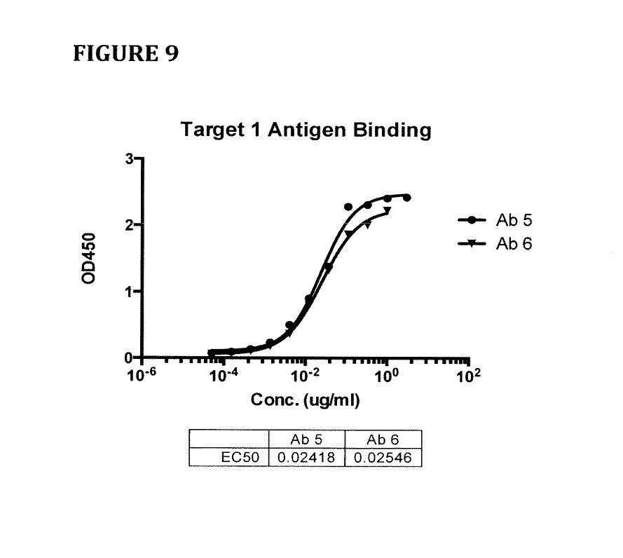

This example demonstrates that multiple anti-CORP antibody sequences were cloned from identified antigen-specific B cells. Exemplary antibodies Ab3 and Ab4 were shown to have high binding affinity for CGRP (see FIG. 9).

### Example 10

### Preparation of Antibodies that Bind Target 1

By using the antibody selection protocol described herein, one can generate a collection of antibodies that exhibit potent functional antagonism of Target 1, including Ab5 and Ab6 which are distinct antibodies with specificity for Target 1.

Immunization Strategy:

Rabbits were immunized with individual peptides as described for CGRPα (see Example 9). Three forms of peptides corresponding to extra-cellular loops of a cell surface protein were designed and synthesized for immunization. These fragments represent likely antibody-accessible epitopes for the intact cellular structure.

ABS Titer Assessment:

Antigen recognition assay was determined for Target 1 by the protocol described in Example 8 for huPCSK9. Rabbits immunized with specific peptides were assayed against that peptide for titer determination.

Tissue Harvesting:

Rabbit spleen, lymph nodes, and whole blood were harvested, processed, and frozen as described above for huPCSK9 (see Example 8).

B Cell Culture:

B cell culture was set as described above for huPCSK9 (see Example 8).

B Cell Culture Antigen Recognition Screening:

Antigen recognition screening was performed as described above as single points (see Example 8).

B Cell Recovery:

The FACS method was performed as described for huPCSK9 (see Example 8).

Amplification and Sequence Determination of Antibody Sequences from Antigen-Specific B Cells:

Antibody sequences were recovered using the method described for huPCSK9 (see Example 8).

Recombinant Production of Monoclonal Antibody of Desired Antigen Specificity and/or Functional Properties:

To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

Antigen-Recognition of Recombinant Antibodies by ELISA:

To characterize recombinant expressed antibodies for their ability to bind to Target 1 peptides antibody-containing solutions were tested by ELISA. See, FIG. 9. All incubations were done at room temperature. Briefly, Neutravidin plates (Pierce) were coated with a biotinylated Target 1 peptide containing solution (1 μg/mL in PBS) for 1 hour. Target 1 peptide-coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antibody being tested for approximately one hour. At the end of this incubation, the plate was washed three times with wash buffer and further incubated with a secondary antibody containing solution (Peroxidase conjugated affinipure F(ab′)2 fragment goat anti-human IgG, Fe fragment specific (Jackson Immunoresearch) for approximately 45 minutes and washed three times. At that point a substrate solution (TMB peroxidase substrate, BioFx) and incubated for 3 to 5 minutes in the dark. The reaction was stopped by addition of a HCl containing solution (0.5M) and the plate was read at 450 nm in a plate-reader.

This example demonstrates that multiple anti-Target 1 antibody sequences were cloned from identified antigen-specific B cells. Exemplary antibodies Ab5 and Ab6 were shown to have high binding affinity for Target 1 (see FIG. 9).

### Example 11

### Preparation of Antibodies that Bind Human Beta-NGF

By using the antibody selection protocol described herein, one can generate an extensive panel of antibodies, including exemplary anti-NGF antibodies Ab7 and Ab8 which are distinct antibodies with specificity for Beta-NGF. The antibodies have high affinity towards NGF (about 10-900 pM K_{D}) and demonstrate potent antagonism of NGF in cell-based screening systems (TF1 and PC-12). Furthermore, the collection of antibodies displayed distinct modes of antagonism toward NGF-driven processes.

Immunization Strategy:

Rabbits were immunized with individual peptides as described above for huPCSK9 (see Example 8).

ABS Titer Assessment:

Antigen recognition assay was determined for huB-NGF by the protocol described above for huPCSK9 (see Example 8).

Functional Titer Assessment:

To identify and characterize antibodies with functional activity, an inhibition of NGF driven proliferation of TF1 (ATCC #CRL-2003) cells was done using CellTiter™ 96 Aqueous One Solution Cell Proliferation Assay (Promega #G3580). Briefly, antibody preparations to be tested were serially diluted in 10% CRPMI (Complete RPMI medium+10% FBS) in a 96 well round bottom polystyrene plate (Costar) with B-NGF. Following an incubation at room temp, antibody-NGF complexes were added to TF1 cells (25,000 cells per well) and incubated for 48 hrs. Following incubation, cell viability was determined using the CellTiter™ 96 Aqueous One Solution Cell Proliferation Assay. Resulting plates were analyzed on a standard plate reader at 492 nm and graphed to establish a proliferation response. Function-modifying titers dampen proliferation in this assay.

Tissue Harvesting:

Rabbit spleen, lymph nodes, and whole blood were harvested, processed, and frozen as described above for huPCSK9 (see Example 8).

B Cell Culture:

B cell culture was set as described for huPCSK9 (see Example 8).

B Cell Culture Antigen Recognition Screening:

Antigen recognition screening was performed as described above (see Example 8) as single points. Master plates were generated based on B-NGF recognition as described above.

Functional Activity Screening:

Master plates were then screened for functional activity in the TF1 proliferation assay as described above.

B Cell Recovery:

The FACS method was performed as described for huPCSK9 (see Example 8).

Amplification and Sequence Determination of Antibody Sequences from Antigen-Specific B Cells:

Antibody sequences were recovered using the method described for huPCSK9 (see Example 8).

Recombinant Production of Monoclonal Antibody of Desired Antigen Specificity and/or Functional Properties:

To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

Antigen-Recognition of Recombinant Antibodies by ELISA:

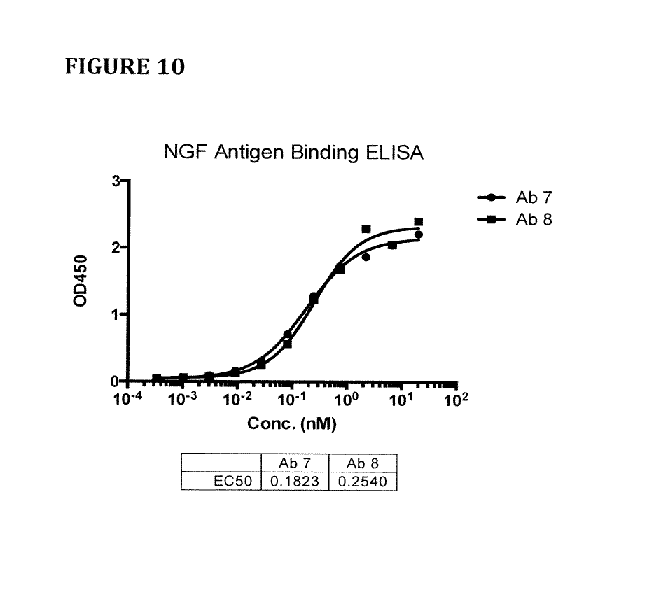

To characterize recombinant expressed antibodies for their ability to bind to hu B-NGF, antibody-containing solutions were tested by ELISA. See, FIG. 10. All incubations were done at room temperature. Briefly, Neutravidin plates (Thermo Scientific) were coated with a biotinylated hu B-NGF containing solution (1 μg/mL in PBS) for 1 hour. Hu B-NGF peptide-coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antibody being tested for approximately one hour. At the end of this incubation, the plate was washed three times with wash buffer and further incubated with a secondary antibody containing solution (Peroxidase conjugated affinipure F(ab′)2 fragment goat anti-human IgG, Fe fragment specific (Jackson Immunoresearch) for approximately 45 minutes and washed three times. At that point a substrate solution (TMB peroxidase substrate, BioFx) and incubated for 3 to 5 minutes in the dark. The reaction was stopped by addition of a HCl containing solution (0.5M) and the plate was read at 450 nm in a plate-reader.

Functional Characterization of Recombinant Antibodies by TF1 Cell Proliferation Assay:

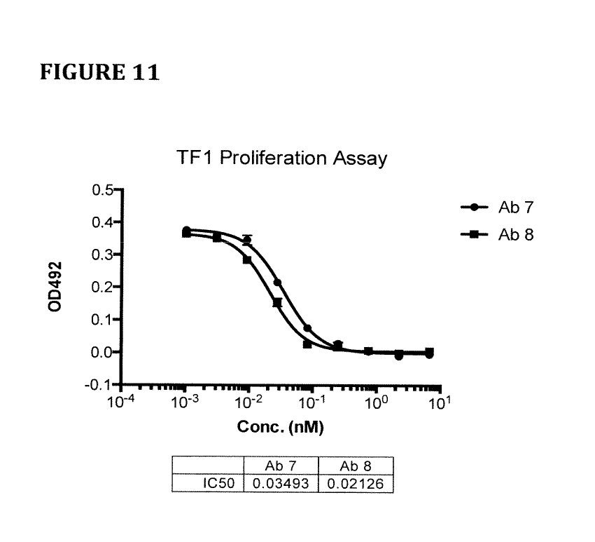

Recombinant hu B-NGF antibodies were assayed for function in the TF-1 proliferation assay as described in the hu B-NGF functional titer section. See, FIG. 11.

This example demonstrates that multiple anti-hu B-NGF antibody sequences were cloned from identified antigen-specific B cells. Exemplary antibodies Ab7 and Ab8 were shown to have high binding affinity for B-NGF (see FIG. 10) and dampen proliferation in the TF1 proliferation assay (see FIG. 11).

### Example 12

### Preparation of Antibodies that Bind Target 2

By using the antibody selection protocol described herein, one can generate an extensive panel of antibodies, including exemplary anti-Target 2 antibodies Ab9 and Ab10 which are distinct antibodies with specificity for Target 2. The antibodies generated can bind a variety of epitopes that include Target 2 specificity as well as retention of binding to homologous proteins to Target 2, and the antibodies were also validated for potency by functional assay.

Immunization Strategy:

Rabbits were immunized with Target 2 as described in Example 9 for CGRPα (mixed with KLH), as well as with additional methods. Specifically Target 2 antigen is a peptide that was directly conjugated to KLH and Rabbit Serum Albumin (RSA). In each instance, as with CGRPα, 100 μg of antigen or KLH/RSA conjugated antigen was used with CFA for the initial immunization and 50 μg boosts for the subsequent immunizations. For conjugated immunizations the μg amount (100 μg initial, 50 μg boost) was matched with free unconjugated antigen. Bleeds were taken in the previously described time points.

ABS Titer Assessment:

Antigen recognition assay was determined for Target 2 by the protocol described for huPCSK9 (see Example 8) using a biotinylated form of Target 2.

Functional Titer Assessment:

Antibodies to Target 2 were validated for potency via a cell based HTRF assay using a secondary messenger readout based on inositol 1-phosphate.

Tissue Harvesting:

Rabbit spleen, lymph nodes, and whole blood were harvested, processed, and frozen as described above for huPCSK9 (see Example 8).

B Cell Culture:

B cell culture was set as described for huPCSK9 (see Example 8).

B Cell Culture Antigen Recognition Screening:

Antigen recognition screening was performed as described above (see Example 8) as single points and master plates were generated from the antigen positive wells.

Functional Activity Screening:

Master plates were then screened for functional activity in the HTRF assay as described above. 70 μl of supernatant was used for the antibody source.

B Cell Recovery:

The FACS method was performed as described for huPCSK9 (see Example 8).

Amplification and Sequence Determination of Antibody Sequences from Antigen-Specific B Cells:

Antibody sequences were recovered using the method described for huPCSK9 (see Example 8).

Recombinant Production of Monoclonal Antibody of Desired Antigen Specificity and/or Functional Properties:

To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

Antigen-Recognition of Recombinant Antibodies by ELISA:

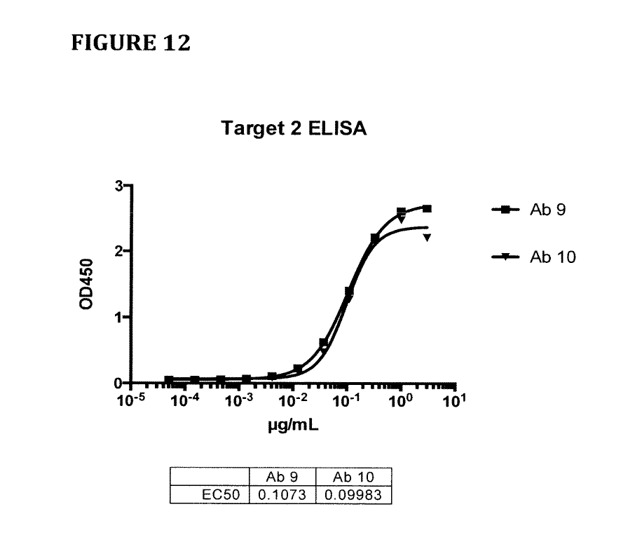

To characterize recombinant expressed antibodies for their ability to bind to Target 2 antibody-containing solutions were tested by ELISA. See, FIG. 12. All incubations were done at room temperature. Briefly, Neutravidin plates (Thermo Scientific) were coated biotinylated Target 2 containing solution (1 μg/mL in PBS) for 1 hour. Target 2 coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antibody being tested for approximately one hour. At the end of this incubation, the plate was washed three times with wash buffer and further incubated with a secondary antibody containing solution (Peroxidase conjugated affinipure F(ab′)₂ fragment goat anti-human IgG, Fc fragment specific (Jackson Immunoresearch) for approximately 45 minutes and washed three times. At that point a substrate solution (TMB peroxidase substrate, BioFx) and incubated for 3 to 5 minutes in the dark. The reaction was stopped by addition of a HCl containing solution (0.5M) and the plate was read at 450 nm in a plate-reader.

Functional Characterization of Recombinant Antibodies by a Cell Based HTRF Assay:

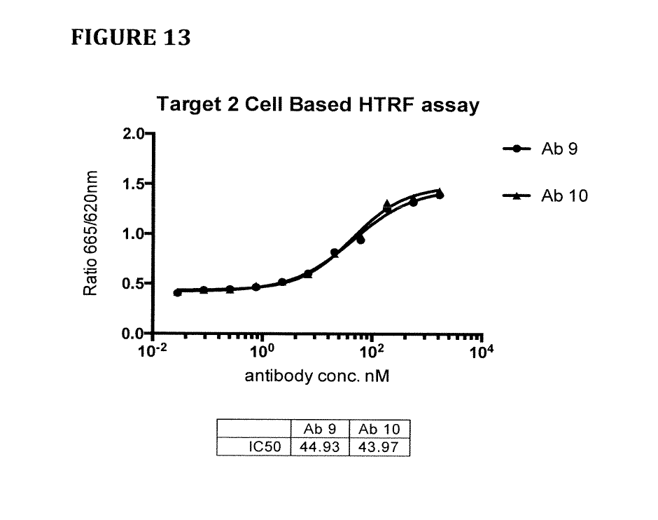

Recombinant Target 2 antibodies were assayed for function in the cell-based IITRF assay as described in the functional titer section. See, FIG. 13.

This example demonstrates that multiple anti-Target 2 antibody sequences were cloned from identified antigen-specific B cells. Exemplary antibodies Ab9 and Ab10 were shown to have high binding affinity for Target 2 (see FIG. 12) and functional activity in the HTRF assay (see FIG. 13).

### Example 13

### Preparation of Antibodies that Bind Target 3

By using the antibody selection protocol described herein, one can generate an extensive panel of antibodies, including exemplary anti-Target 3 antibodies Ab11 and Ab12 which are distinct antibodies with specificity for Target 3. The antibodies generated bind a variety of epitopes specific to Target 3.

Immunization Strategy: Rabbits were immunized with Target 3 as described in Example 9 for CGRPα (mixed with KLH), as well with additional methods. Specifically Target 3 antigen is a peptide that was directly conjugated to KLH. In each instance, as with CGRPα, 100 μg of antigen or KLH/RSA conjugated antigen was used with CFA for the initial immunization and 50 μg boosts for the subsequent immunizations. For conjugated immunizations the μg amount (100 μg initial, 50 μg boost) was matched with free unconjugated antigen. Bleeds were taken in the previously described time points.

ABS Titer Assessment: Antigen recognition assay was determined for Target 3 by the protocol described for huPCSK9 (see Example 8) using a biotinylated form of Target 3.

Functional Titer Assessment: Antibodies to Target 3 can be validated for potency via a cell-based electrochemiluminescence (Meso Scale Discovery, MSD) assay using a secondary messenger readout based on cyclic-AMP.

Tissue harvesting: Rabbit spleen, lymph nodes, and whole blood were harvested, processed, and frozen as described above for huPCSK9 (see Example 8).

B cell culture: B cell culture was set as described above for huPCSK9 (see Example 8).

B cell culture Antigen Recognition Screening: Antigen recognition screening may be performed as described above (see Example 8) as single points and master plates were generated from the antigen positive wells.

Functional Activity Screening: Master plates may be screened for functional activity in the MSD assay as described above. 70 μl of supernatant may be used for the antibody source.

B Cell Recovery: The FACS method was performed as described above for huPCSK9 (see Example 8).

Amplification and sequence determination of Antibody Sequences From Antigen-Specific B Cells: Antibody sequences were recovered using the method described for huPCSK9 (see Example 8).

Recombinant Production of Monoclonal Antibody of Desired Antigen Specificity and/or Functional Properties: To determine antigen specificity and functional properties of recovered antibodies from specific B-cells, vectors driving the expression of the desired paired heavy and light chain sequences were transfected into human kidney two-hundred and ninety-three cells and recombinant antibody was subsequently recovered from the culture medium.

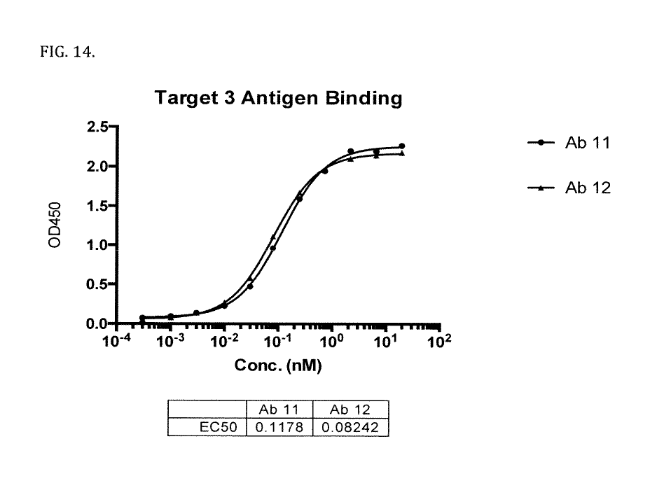

Antigen-recognition of recombinant antibodies by ELISA: To characterize recombinant expressed antibodies for their ability to bind to Target 3 antibody-containing solutions were tested by ELISA. See FIG. 14. All incubations were done at room temperature. Briefly, Neutravidin plates (Thermo Scientific) were coated biotinylated Target 3 containing solution (1 μg/mL in PBS) for 1 hour. Target 3 coated plates were then washed three times in wash buffer (PBS, 0.05% Tween-20). The plates were then blocked using a blocking solution (PBS, 0.5% fish skin gelatin, 0.05% Tween-20) for approximately one hour. The blocking solution was then removed and the plates were then incubated with a dilution series of the antibody being tested for approximately one hour. At the end of this incubation, the plate was washed three times with wash buffer and further incubated with a secondary antibody containing solution (Peroxidase conjugated affinipure F(ab′)2 fragment goat anti-human IgG, Fe fragment specific (Jackson Immunoresearch) for approximately 45 minutes and washed three times. At that point a substrate solution (TMB peroxidase substrate, BioFx) and incubated for 3 to 5 minutes in the dark. The reaction was stopped by addition of a HCl containing solution (0.5M) and the plate was read at 450 nm in a plate-reader.

Functional characterization of recombinant antibodies by a cell based HTRF or MSD assay: Recombinant Target 3 antibodies can be assayed for function in the cell-based HTRF assay as described in the functional titer section.

This example demonstrates that multiple anti-Target 3 antibody sequences were cloned from identified antigen-specific B cells. Exemplary antibodies Ab11 and Ab12 were shown to have high binding affinity for Target 3 (see FIG. 14).

The foregoing examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

The invention may be practiced in ways other than those particularly described in the foregoing description and examples. Numerous modifications and variations of the invention are possible in light of the above teachings and, therefore, are within the scope of the appended claims.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The entire disclosure of each document cited herein (including patents, patent applications, journal articles, abstracts, manuals, books, or other disclosures), including each document cited in the Background, Summary, Detailed Description, and Examples, is hereby incorporated by reference herein in its entirety.

What is claimed is:

1. A method for identifying a B cell that expresses an antigen-specific antibody, comprising:
(i) obtaining B cells from a host that has been immunized or exposed naturally to an antigen of interest;
(ii) enriching a fraction of said B cells to obtain an enriched population of antigen-specific B cells, which contains a greater percentage of B cells that produce an antibody that binds to the antigen of interest relative to the B cell fraction prior to enrichment;
(iii) separately culturing one or more fractions from said enriched antigen-specific B cell population under culture conditions that favor the formation of a clonal B cell population that produces a single antibody that binds to the antigen of interest;
(iv) detecting the clonal B cell population that produces a single antibody that binds to the antigen of interest, thereby identifying one or more antigen-specific B cells;
(v) optionally screening the clonal antigen-specific B cell population identified in step (iv) to identify B cells that produce an antigen-specific antibody possessing at least one desired functional property;
(vi) optionally pooling antigen-specific B cells obtained from different clonal B cell cultures;
(vii) staining the antigen-specific B cells obtained after step (iv) or after optional step (v) or optional step (vi) said with at least one label that facilitates positive and/or negative selection of the stained B cells; and
(viii) sorting the stained antigen-specific B cells and optionally gating the sorted stained B cells to isolate a single antigen-specific B cell.

2. The method of claim 1, further comprising cloning the antigen-specific antibody variable sequences encoding the variable light chain region and/or the variable heavy chain region by:
(ix) placing the sorted B cells into a reverse transcription polymerase chain reaction (RT-PCR) reaction medium that facilitates the amplification of antigen-specific antibody variable sequences expressed by the sorted B cells, wherein optionally step (xi) comprises expression in a recombinant cell, such as a yeast, bacterium, plant, insect, amphibian or mammalian cell; a diploid yeast, a Pichia species; or Pichia pastoris;
(x) sequencing the amplified nucleic acids encoding the antigen-specific antibody variable sequences;
(xi) expressing the amplified nucleic acids or a variant thereof encoding the antigen-specific antibody variable sequences to produce antibody polypeptides; and
(xii) determining which of the expressed antibody polypeptides bind to the antigen of interest; optionally by determining which of the expressed antibody polypeptides bind to the antigen of interest using radioimmunoassay (RIA), enzyme-linked immunoadsorbent assay (ELISA), immunoprecipitation, fluorescent immunoassays, western blot, surface plasmon resonance (BIAcore®) analysis or another antigen binding assay, such as by ELISA.

3. The method of claim 1, wherein the host is a guinea pig, rabbit, mouse, rat, non-human primate or human, or wherein the host is a rabbit.

4. The method of any one of claims 1-3, wherein step (i) comprises harvesting B cells from at least one source selected from spleen, lymph node, bone marrow, peripheral blood mononuclear cells and blood, or wherein step (i) comprises harvesting B cells from more than one source selected from spleen, lymph node, bone marrow, peripheral blood mononuclear cells and blood and pooling said B cells from more than one source.

5. The method of any one of claims 1-6, further comprising establishing a titer of antigen-specific and/or neutralizing antibodies present in sera from the host.

6. The method of claim 1 or 2, wherein:
(a) the enrichment step (ii) comprises affinity purification of antigen-specific B cells using an antigen directly or indirectly attached to a solid matrix or support, wherein optionally the solid matrix comprises magnetic beads, optionally the solid matrix comprises a column, and/or optionally the antigen that is directly or indirectly attached to the solid matrix or support is biotinylated and attached to the matrix or support via streptavidin, avidin or neutravidin;
(b) said enrichment step (ii) comprises: (1) combining B cells with biotin-labeled antigen; (2) optionally washing the B cell/biotin-labeled antigen composition; (3) introducing streptavidin beads to the B cell/biotin-labeled antigen composition of (1) or (2); (4) passing the streptavidin beads/B cell/biotin-labeled antigen composition over a column; and (5) washing the column and eluting the bound B cells from the column, thereby obtaining an enriched antigen-specific B cell population;
(c) said enrichment step (ii) comprises: (1) combining biotin-labeled antigen with streptavidin beads; (2) passing the biotin-labeled antigen/streptavidin bead composition over a column; (3) washing the column and eluting biotin-labeled antigen-coated beads from the column; (4) combining B cells with the coated beads; (5) passing the mixture of B cells and coated beads over the column; and (6) washing the column and eluting the bound B cells from the column, thereby obtaining an enriched antigen-specific B cell population; or
(d) said enrichment method (a), (b), and/or (c) or a combination of said enrichment methods, which is repeated at least once thereby resulting in a further enriched antigen-specific B cell population;
wherein the enrichment step (ii) enriches the percentage of antigen-specific B cells by at least 2-fold, at least 5-fold, at least 10-fold, at least 50-fold, at least 100-fold, at least 1,000-fold or at least 10,000-fold, and/or wherein the percentage of antigen-specific B cells in the enriched B cell population is at least 1%, 5%, or 10%.

7. The method of any one of the foregoing claims, wherein the enriched antigen-specific B cells are cultured in step (iii) in a medium comprising feeder cells, wherein optionally:
(a) the feeder cells are irradiated EL4 cells;
(b) the culture medium comprises activated T cell conditioned medium;
(c) the enriched B cells are cultured in a medium comprising between about 1% and about 5% activated rabbit T cell conditioned medium;
wherein optionally said culturing is effected for at least about 1-9 days, 2-8 days, 3-7 days, 4-6 days, or 5-7 days, or optionally said culturing is effected for about 5-7 days.

8. The method of any one of the foregoing claims, wherein said enriched B cells are cultured in a multi-well plate with each well containing at least 1, at least 10, at least 25, at least 50, at least 100 or at least 200 enriched B cells; and optionally
(a) said enriched B cells are cultured in a multi well plate with each well containing about 50 to about 100 enriched B cells;
(b) said enriched B cells are cultured in a multi-well plate with each well containing about 25 to about 50 enriched B cells;
(c) said enriched B cells are cultured in a multi-well plate with each well containing about 10 to about 25 enriched B cells; or
(d) said enriched B cells are cultured in a multi-well plate with each well containing about 1 to about 200 of the enriched antigen-specific B cells combined with irradiated EL4 cells and T cell supernatant (TSN) in each well of a multi-well plate.

9. The method of any one of the foregoing claims, wherein the antigen-recognition detection step (iv) comprises removing supernatant from the cultured enriched B cells and assaying said supernatant to identify the individual wells in the multi-well plate that contain antigen-reactive supernatants thereby detecting wells containing antigen-specific B cells, wherein optionally:
(a) the supernatant is evaluated by ELISA;
(b) the supernatant is assayed for antigen-specific IgG production and total IgG production after culturing the enriched B cells for about 2 to about 7 days;
(c) the supernatant is assayed for total IgG production by (1) coating plates with an anti-species Fab; (2) adding supernatant from cultured B cells to the plate; and (3) detecting the total IgG in the supernatant with an anti-species IgG, wherein optionally the anti-species Fab is an anti-rabbit Fab and the anti-species IgG is an anti-rabbit IgG; and/or
(d) the supernatant is assayed for antigen-specific IgG production by (1) coating plates with unlabeled antigen or coating streptavidin plates with biotin-labeled antigen; (2) adding supernatant from cultured B cells to the plate; and (3) detecting the antigen-specific IgG in the supernatant with an anti-species IgG; wherein optionally wherein the anti-species IgG is an anti-rabbit IgG; wherein optionally the ratio of antigen-specific wells to total IgG wells in the multi-well plate correlates with B cell enrichment and clonality.

10. The method of any one of claims 28 to 35, wherein the optional functional activity screening step (v) comprises assaying the antigen-reactive supernatants using an antigen-specific functional assay to identify wells that contain antigen-specific B cells that secrete antigen-specific antibodies having at least one desired functional property; wherein optionally:
(a) the optional functional activity screening step (v) comprises screening the antigen-specific B cells identified in step (iv) to identify B cells that produce an antigen-specific antibody that exhibits agonism or antagonism of antigen binding to a binding partner; induction or inhibition of the proliferation of a specific target cell type; induction or inhibition of lysis of a target cell; or induction or inhibition of a biological pathway involving the antigen; and further optionally:
(b) the antigen-specific antibody is screened for induction or inhibition of the proliferation of T1165 cells; induction or inhibition of the proliferation of TF1 cells; induction or inhibition of cAMP production in SK-N-MC cells; or inhibition of PCSK9/LDLR interaction.

11. The method of any one of the foregoing claims, wherein:
(a) antigen-reactive supernatants from the ELISA screen are transferred to another plate and frozen; and/or
(b) one or more freezing and storage steps intervening one or more of the method steps, optionally with the addition of a freezing or storage medium.

12. The method of any one of the foregoing claims, wherein the staining step (vii) facilitates a negative antigen-specific B selection method, wherein optionally said negative antigen-specific B selection method comprises sorting all viable, non-EL4 cells using flow cytometry; wherein optionally the negative antigen-specific B selection is effected by staining B cells with a first label that stains irradiated EL4 cells, such as a labeled antibody specific for Thy1.2, and a second label that stains non-viable cells, such as propidium iodide (PI).

13. The method of any one of the foregoing claims, wherein the staining step (vii) facilitates a positive antigen-specific B selection method, wherein optionally said positive antigen-specific B selection method comprises sorting all viable, species-specific B cells using flow cytometry; wherein optionally the positive antigen-specific B selection is effected by staining with a first label that stains species-specific B cells, such as a labeled antibody specific for a species IgG such as an anti-rabbit IgG, and a second label that stains non-viable cells, such as propidium iodide (PI).

14. The method of any one of the foregoing claims, wherein the optional gating step (viii) comprises selecting sorted viable, non-EL4 cells that possess a distinct physical profile (FSC/SSC population).

15. The method of any one of the foregoing claims, wherein flow cytometry is performed using fluorescence-activated cell sorting (FACS) or immunomagnetic cell sorting (MACS).

16. The method of any one of the foregoing claims, wherein the sorting step (viii) comprises sorting the cells directly into RT-PCR reaction medium.

17. The method of any of the previous claims, wherein (a) different individual wells containing antigen-specific B cells secreting antigen-specific antibodies are combined prior to staining and cell sorting, or (b) different individual wells containing antigen-specific B cells that secrete antigen-specific antibodies having similar affinity and/or desired functional properties are combined prior to staining and sorting; wherein optionally:
(i) antigen-specific B cells from about 2 to about 10 different individual wells are combined;
(ii) antigen-specific B cells from about 10 to about 50 different individual wells are combined; or
(iii) antigen-specific B cells from about 50 to about 150 different individual wells are combined.

18. The method of any one of the foregoing claims, wherein step (i) comprises obtaining B cells from the host at about 20 to about 90 days after immunization, such as at about 50 to about 60 days after immunization.

19. The method of any one of the preceding claims, wherein the optional gating step comprises constructing a gate based on auto-fluorescence of unstained cells.

20. A sorted population of predominantly viable, non-EL4 cells produced according to the method of any of the foregoing claims that possess a distinct physical profile (FSC/SSC population), wherein optionally said population comprises viable, species-specific B cells.

21. The sorted population of cells according to claim 20, which is obtained by flow cytometry using a negative antigen-specific B selection which is effected by staining B cells with a first label that stains irradiated EL4 cells, such as a labeled antibody specific for Thy1.2, and a second label that stains non-viable cells, such as propidium iodide (PI).

22. The sorted population of claim 20, which is obtained by flow cytometry using a positive antigen-specific B selection, which is effected by staining with a first label that stains species-specific B cells, such as a labeled antibody specific for rabbit IgG and a second label that stains non-viable cells, such as propidium iodide (PI).

23. The sorted population of any one of claims 20-22, which comprise B cells specific to a human antigen, such as a tumor antigen, CGRP, NGF, a neurotransmitter, PCSK9, or IL-6.


SEQUENCE LISTING (13 sequences)

SEQ ID NO: 1 - DNA, 20 bp, Artificial
  PCR primer
agnacccagcatggacanna

SEQ ID NO: 2 - DNA, 39 bp, Artificial
  PCR primer
gatatcaagcttcgaatcgacatggacacgagggccccc

SEQ ID NO: 3 - DNA, 25 bp, Artificial
  PCR primer
gganngnatttattngccacncaca

SEQ ID NO: 4 - DNA, 35 bp, Artificial
  PCR primer
tctagacgtacgtttgaccaccacctcggtccctc

SEQ ID NO: 5 - DNA, 33 bp, Artificial
  PCR primer
tctagacgtacgtaggatctccagctcggtccc

SEQ ID NO: 6 - DNA, 36 bp, Artificial
  PCR primer
tctagacgtacgtttgatttccacattggtgccagc

SEQ ID NO: 7 - DNA, 20 bp, Artificial
  PCR primer
agacnctcaccatggagact

SEQ ID NO: 8 - DNA, 34 bp, Artificial
  PCR primer
gatatcaagcttacgctcaccatggagactgggc

SEQ ID NO: 9 - DNA, 17 bp, Artificial
  PCR primer
actggctccgggaggta

SEQ ID NO: 10 - DNA, 37 bp, Artificial
  PCR primer
cgcgcgctcgagacggtgacnagggtnccnnggcccc

SEQ ID NO: 11 - DNA, 23 bp, Artificial
  PCR primer
gcgcgccaccagacataatagct

SEQ ID NO: 12 - DNA, 23 bp, Artificial
  PCR primer
agcccaaggtcaccgtgctagag

SEQ ID NO: 13 - DNA, 27 bp, Artificial
  PCR primer
gtatttattcgccacacacacacgatg